SEQUENCE LISTING (55 sequences)

SEQ ID NO: 1 - protein, 8 aa, synthetic construct
GDTSSIFL

SEQ ID NO: 2 - protein, 8 aa, synthetic construct
INHEGTTN

SEQ ID NO: 3 - protein, 9 aa, synthetic construct
IARSIFGNS

SEQ ID NO: 4 - protein, 25 aa, synthetic construct
QVQLVESGGGLVQAGGSLTLSCAAS

SEQ ID NO: 5 - protein, 17 aa, synthetic construct
MGWFRQAPGKQRDMVAL

SEQ ID NO: 6 - protein, 37 aa, synthetic construct
YKDSVKGRFAISRDNAKNTVSLQMNSLTPEDTAVYFC

SEQ ID NO: 7 - protein, 11 aa, synthetic construct
WGQGTQVTVSS

SEQ ID NO: 8 - protein, 115 aa, synthetic construct
QVQLVESGGGLVQAGGSLTLSCAASGDTSSIFLMGWFRQAPGKQRDMVALINHEGTTNYKDSVKGRFAISRDNAKNTVSLQMNSLTPEDTAVYFCIARSIFGNSWGQGTQVTVSS

SEQ ID NO: 9 - protein, 144 aa, synthetic construct
MAQVQLVESGGGLVQAGGSLTLSCAASGDTSSIFLMGWFRQAPGKQRDMVALINHEGTTNYKDSVKGRFAISRDNAKNTVSLQMNSLTPEDTAVYFCIARSIFGNSWGQGTQVTVSSGPGGQREARHHHHHHGAYPYDVPDYAS

SEQ ID NO: 10 - DNA, 435 bp, synthetic construct
  source 1..435: mol_type = other DNA
atggctcaggtgcagctcgtggagtctgggggaggcttggtgcaggctggggggtctctgacactctcctgtgcagcctctggagacacctcgagtatctttctgatgggctggttccgccaggctccggggaagcagcgcgacatggtcgcacttattaatcatgaaggtaccacaaactataaagactccgtgaagggccgattcgccatctccagagacaacgccaagaacaccgtctctctacaaatgaacagcctcacacctgaggacacggccgtctatttttgtattgctcgctctatttttggaaattcctggggccaggggacccaggtcaccgtctccagcggcccgggaggccaacgggaggccagacaccatcaccaccatcatggcgcatatccgtatgatgtgccggactatgcttcttag

SEQ ID NO: 11 - protein, 8 aa, synthetic construct
GDTSSIFL

SEQ ID NO: 12 - protein, 8 aa, synthetic construct
INYDGTTN

SEQ ID NO: 13 - protein, 9 aa, synthetic construct
NARSIFGNS

SEQ ID NO: 14 - protein, 25 aa, synthetic construct
QVQLVESGGGLVQAGGSLTLSCAAF

SEQ ID NO: 15 - protein, 17 aa, synthetic construct
MGWFRQAPGKQRDMVAL

SEQ ID NO: 16 - protein, 37 aa, synthetic construct
YKDTVKGRFAISRDNAKNTVSLQMNSLSPEDTAVYFC

SEQ ID NO: 17 - protein, 11 aa, synthetic construct
WGQGTQVTVSS

SEQ ID NO: 18 - protein, 115 aa, synthetic construct
QVQLVESGGGLVQAGGSLTLSCAAFGDTSSIFLMGWFRQAPGKQRDMVALINYDGTTNYKDTVKGRFAISRDNAKNTVSLQMNSLSPEDTAVYFCNARSIFGNSWGQGTQVTVSS

SEQ ID NO: 19 - protein, 144 aa, synthetic construct
MAQVQLVESGGGLVQAGGSLTLSCAAFGDTSSIFLMGWFRQAPGKQRDMVALINYDGTTNYKDTVKGRFAISRDNAKNTVSLQMNSLSPEDTAVYFCNARSIFGNSWGQGTQVTVSSGPGGQREARHHHHHHGAYPYDVPDYAS

SEQ ID NO: 20 - DNA, 435 bp, synthetic construct
  source 1..435: mol_type = other DNA
atggctcaggtgcagctcgtggagtctgggggaggcttggtgcaggcaggggggtctctgacactctcctgtgcagcctttggagacacctcgagtatctttctgatgggctggttccgccaggctccggggaagcagcgcgacatggtcgcacttattaattatgatggtaccacaaactataaagacaccgtgaagggccgattcgccatctccagagacaacgctaagaacaccgtctctctacaaatgaacagcctctcacctgaggacacggccgtctatttttgtaacgctcgctctatttttggaaattcctggggccaggggacccaggtcaccgtctccagcggcccgggaggccaacgggaggccagacaccatcaccaccatcatggcgcatatccgtatgatgtgccggactatgcttcttag

SEQ ID NO: 21 - protein, 140 aa, synthetic construct
MAQVQLVESGGGLVQAGGSLTLSCAASGDTSSIFLMGWFRQAPGKQRDMVALINHDGTTNYKDSVKGRFAISRDNAKNTVSLQMNSLTPEDTAVYFCNARSIFGNSWGQGTQVTVSSGPGGQHHHHHHGAYPYDVPDYAS

SEQ ID NO: 22 - DNA, 423 bp, synthetic construct
  source 1..423: mol_type = other DNA
atggctcaggtgcagctcgtggagtctgggggaggcttggtgcaggctggggggtctctgacactctcctgtgcagcctctggagacacctcgagtatctttctgatgggctggttccgccaggctccggggaagcagcgcgacatggtcgcacttattaatcatgatggtaccacaaactataaagactccgtgaagggccgattcgccatctccagagacaacgccaagaacaccgtctctctacaaatgaacagcctcacacctgaggacacggccgtctatttttgtaatgctcgctctatttttggaaattcctggggccaggggacccaggtcaccgtctccagcggcccgggaggccaacaccatcaccaccatcatggcgcatatccgtatgatgtgccggactatgcttcttag

SEQ ID NO: 23 - protein, 8 aa, synthetic construct
  VARIANT 3: note = "H" or "Y"
  VARIANT 4: note = "E" or "D"
INXXGTTN

SEQ ID NO: 24 - protein, 9 aa, synthetic construct
  VARIANT 1: note = "I" or "N"
XARSIFGNS

SEQ ID NO: 25 - protein, 25 aa, synthetic construct
  VARIANT 25: note = "S" or "F"
QVQLVESGGGLVQAGGSLTLSCAAX

SEQ ID NO: 26 - protein, 37 aa, synthetic construct
  VARIANT 4: note = "S" or "T"
  VARIANT 28: note = "T" or "S"
YKDXVKGRFAISRDNAKNTVSLQMNSLXPEDTAVYFC

SEQ ID NO: 27 - DNA, 1704 bp, synthetic construct
  source 1..1704: mol_type = other DNA
atggccttaccagtgaccgccttgctcctgccgctggccttgctgctccacgccgccaggccggctcaggtgcagctcgtggagtctgggggaggcttggtgcaggctggggggtctctgacactctcctgtgcagcctctggagacacctcgagtatctttctgatgggctggttccgccaggctccggggaagcagcgcgacatggtcgcacttattaatcatgaaggtaccacaaactataaagactccgtgaagggccgattcgccatctccagagacaacgccaagaacaccgtctctctacaaatgaacagcctcacacctgaggacacggccgtctatttttgtattgctcgctctatttttggaaattcctggggccaggggacccaggtcaccgtctccagcggcccgggaggccaacgggaggccagacaccatcaccaccatcatggcgcatatccgtatgatgtgccggactatgcttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgggcaccagcctgctgtgctggatggccctgtgcctgctgggcgccgaccacgccgatgcctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtagcggagggggaggcagcccagcccccagacctcccaccccagcccccaccatcgccagccagcctctgagcctgagacccgaggcctgccgcccagccgccggcggcgccgtgcacaccagaggcctggatttcgcctgcgatatctacatctgggccccactggccggcacctgtggcgtgctgctgctgagcctggtgatcaccctgtactgcaaccaccgcaaccgcaggcgcgtgtgcaagtgccccaggcccgtggtg

SEQ ID NO: 28 - protein, 7 aa, synthetic construct
GLTFSSY

SEQ ID NO: 29 - protein, 5 aa, synthetic construct
WSGRD

SEQ ID NO: 30 - protein, 13 aa, synthetic construct
NPWPVAAPRSGTY

SEQ ID NO: 31 - protein, 25 aa, synthetic construct
QVQLVESGGGLVQPGGSLRLSCAAS

SEQ ID NO: 32 - protein, 20 aa, synthetic construct
NMGWFRQAPGQGLEAVASIT

SEQ ID NO: 33 - protein, 41 aa, synthetic construct
TFYADSVKGRFTISRDNSKNTLYLQMNSLRAEDTAVYYCAA

SEQ ID NO: 34 - protein, 11 aa, synthetic construct
WGQGTLVTVSS

SEQ ID NO: 35 - protein, 22 aa, synthetic construct
SGGGGSGGGGSGGGGSGGGGSG

SEQ ID NO: 36 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 37 - protein, 18 aa, synthetic construct
GSTSGSGKPGSGEGSTKG

SEQ ID NO: 38 - protein, 405 aa, synthetic construct
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGAQVQLVESGGGLVQAGGSLTLSCAAFGDTSSIFLMGWFRQAPGKQRDMVALINYDGTTNYKDTVKGRFAISRDNAKNTVSLQMNSLSPEDTAVYFCNARSIFGNSWGQGTQVTVSSVDEHHHHHHHHHH

SEQ ID NO: 39 - protein, 392 aa, synthetic construct
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGAQVQLVESGGGLVQAGGSLTLSCAAFGDTSSIFLMGWFRQAPGKQRDMVALINYDGTTNYKDTVKGRFAISRDNAKNTVSLQMNSLSPEDTAVYFCNARSIFGNSWGQGTQVTVSS

SEQ ID NO: 40 - DNA, 366 bp, synthetic construct
  source 1..366: mol_type = other DNA
caggtccaactcgtagagtctggcggtggattggtgcagcctggcggtagcttgcggctctcctgtgccgcatcaggattgacatttagttcttacaatatggggtggttccgccaagctccgggacagggtcttgaagcagtcgcctctattacatggtcaggaagagacaccttctatgcagactccgtaaaaggaaggtttacgatctcacgggataactctaaaaacaccttgtatcttcagatgaactccctgagagccgaggacacagccgtgtactattgtgccgctaacccatggccagtcgcggcaccccggagcggcacatattggggacaaggtacacttgtaactgttagctcc

SEQ ID NO: 41 - DNA, 342 bp, synthetic construct
  source 1..342: mol_type = other DNA
aattgggttaatgtcattagtgaccttaagaagatcgaagacctgatccaaagtatgcacattgatgccactctctatactgaatccgacgtacaccctagttgtaaagtaacggccatgaaatgttttttgttggagctgcaagtcatttccttggagtcaggcgatgccagcatccatgatacagtggaaaatctcataatcctcgctaacaactcactttctagtaatggaaatgtgactgaaagcgggtgtaaggaatgtgaagaactcgaagaaaaaaatattaaagagttcctgcagagcttcgtacacattgtacagatgttcataaataccagc

SEQ ID NO: 42 - DNA, 348 bp, synthetic construct
  source 1..348: mol_type = other DNA
gctcaagtgcagttggtggagtcaggcggaggactcgtacaagcgggaggatcactcaccctttcatgcgcagcgtttggtgacacgtcatccatttttctcatgggctggtttcggcaagccccgggcaagcaacgagatatggtcgcgcttattaactacgatgggactacaaattacaaggacactgttaagggccgcttcgccatttcacgcgacaatgccaaaaatacggtaagtttgcagatgaatagtcttagcccagaagatactgctgtctacttttgcaacgcaaggagtatcttcggaaattcctggggccaaggcacgcaggttactgtttccagt

SEQ ID NO: 43 - protein, 20 aa, synthetic construct
PSGQAGAAASESLFVSNHAY

SEQ ID NO: 44 - protein, 7 aa, synthetic construct
EASGGPE

SEQ ID NO: 45 - protein, 10 aa, synthetic construct
GGGGSGGGGS

SEQ ID NO: 46 - protein, 568 aa, synthetic construct
MALPVTALLLPLALLLHAARPAQVQLVESGGGLVQAGGSLTLSCAASGDTSSIFLMGWFRQAPGKQRDMVALINHEGTTNYKDSVKGRFAISRDNAKNTVSLQMNSLTPEDTAVYFCIARSIFGNSWGQGTQVTVSSGPGGQREARHHHHHHGAYPYDVPDYASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 47 - protein, 21 aa, synthetic construct
MALPVTALLLPLALLLHAARP

SEQ ID NO: 48 - protein, 143 aa, synthetic construct
AQVQLVESGGGLVQAGGSLTLSCAASGDTSSIFLMGWFRQAPGKQRDMVALINHEGTTNYKDSVKGRFAISRDNAKNTVSLQMNSLTPEDTAVYFCIARSIFGNSWGQGTQVTVSSGPGGQREARHHHHHHGAYPYDVPDYAS

SEQ ID NO: 49 - protein, 45 aa, synthetic construct
TTTPAPRPPTPAPTIASQPLSLRPEACRPAAGGAVHTRGLDFACD

SEQ ID NO: 50 - protein, 24 aa, synthetic construct
IYIWAPLAGTCGVLLLSLVITLYC

SEQ ID NO: 51 - protein, 42 aa, synthetic construct
KRGRKKLLYIFKQPFMRPVQTTQEEDGCSCRFPEEEEGGCEL

SEQ ID NO: 52 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 24 aa, synthetic construct
LEGGGEGRGSLLTCGDVEENPGPR

SEQ ID NO: 54 - protein, 157 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 55 - protein, 122 aa, synthetic construct
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

(19) United States
(12) Patent Application Publication
(10) Pub. No.: US 2026/0337266 A1
(43) Pub. Date: Oct. 29, 2026

(54) CD83 Binding Proteins

(71) Applicant: REGENTS OF THE UNIVERSITY OF MINNESOTA, Minneapolis, MN (US)

(72) Inventors: Brian Betts, Minneapolis, MN (US); Aaron LeBeau, Madison, WI (US); Martin Felices, Minneapolis, MN (US); Jeffrey T. Miller, Naperville, IL (US); Mehrdad Hefazi Torghabeh, Minneapolis, MN (US); Bruce Blazar, Minneapolis, MN (US)

(21) Appl. No.: 19/104,851

(22) PCT Filed: Aug. 18, 2023
(86) PCT No.: PCT/US23/72480

§ 371 Date: Feb. 19, 2025

Related U.S. Application Data
(60) Provisional application No. 63/399,421, filed on Aug. 19, 2022.

(51) Int. Cl. C07K 16/28; A61K 40/11; A61K 40/31; A61K 40/42; A61P 35/00; C07K 14/54

(52) U.S. Cl. CPC C07K 16/2803; A61K 40/11; A61K 40/31; A61K 40/421; A61P 35/00; C07K 14/5443; C07K 16/283; C07K 2317/22; C07K 2317/31; C07K 2317/565; C07K 2317/569

(57) ABSTRACT

The disclosure provides a CD83 binding protein comprising the amino acid sequences of SEQ ID NO: 1, SEQ ID NO: 23, and SEQ ID NO: 24. A multispecific construct comprising an NK cell engaging domain, an NK activating domain operably linked to the NK engaging domain, and a targeting domain comprising the CD83 binding protein operably linked to the NK activating domain and the NK engaging domain also is provided. The disclosure further provides a chimeric antigen receptor (CAR) comprising an extracellular domain comprising the CD83 binding protein, a transmembrane domain, and an intracellular signaling domain.

### CROSS REFERENCE TO RELATED APPLICATIONS AND INCORPORATION BY REFERENCE

This application claims the benefit of U.S. Provisional Patent Application No. 63/399,421, filed Aug. 19, 2022, which is hereby incorporated by reference.

Incorporated by reference in its entirety is a computer-readable nucleotide/amino acid sequence listing submitted concurrently herewith and identified as follows: XML file named “57097_Seqlisting.xml”, 57,868 bytes, created Aug. 3, 2023.

### STATEMENT OF GOVERNMENT INTEREST

This invention was made with government support under CA065493 and HL118979 awarded by the National Institutes of Health. The government has certain rights in the invention.

### FIELD

This disclosure relates CD83 binding proteins and methods of use.

### BACKGROUND

CD83 is a 45 kDa, type-I membrane glycoprotein which is a member of the immunoglobulin superfamily. CD83 is expressed on the surface of activated human dendritic cells (DCs) as well as allo-activated T cells. As such, CD83 is an attractive target for immunotherapies with the potential of eliminating inflammatory DCs, alloreactive donor T cells, and cancer cells. Therapeutics which target CD83 show great promise in the treatment and prevention of cancer and immunological disorders, including Graft versus Host Disease (GvHD) and other autoimmune conditions.

### SUMMARY

The disclosure provides a CD83 binding protein comprising the amino acid sequences of SEQ ID NO: 1, SEQ ID NO: 23, and SEQ ID NO: 24. In various aspects, the binding protein comprises the amino acid sequences of SEQ ID NOs: 1-3 or the amino acid sequences of SEQ ID NOs: 11-13. Optionally, the binding protein comprises the amino acid sequence of SEQ ID NO: 25, such as the amino acid sequence of SEQ ID NO: 4 or SEQ ID NO: 14; and/or the binding protein comprises the amino acid sequence of SEQ ID NO: 5; and/or the binding protein comprises the amino acid sequence of SEQ ID NO: 26, such as the amino acid sequence of SEQ ID NO: 6 or the amino acid sequence of SEQ ID NO: 16; and/or the binding protein comprises the amino acid sequence of SEQ ID NO: 7. For example, the disclosure provides a CD83 binding protein comprising the amino acid sequence of SEQ ID NO: 8 (e.g., the amino acid sequence of SEQ ID NO: 9). The disclosure further provides a CD83 binding protein comprising the amino acid sequence of SEQ ID NO: 18 (e.g., the amino acid sequence of SEQ ID NO: 19).

A nucleic acid molecule comprising a nucleotide sequence encoding the CD83 binding protein, a host cell comprising the nucleic acid, a method of making the CD83 binding protein, and a composition comprising the CD83 binding protein also are provided.

Also provided is a multispecific construct comprising an NK cell engaging domain, an NK activating domain operably linked to the NK engaging domain, and a targeting domain comprising the CD83 binding protein operably linked to the NK activating domain and the NK engaging domain. In various aspects, the NK engaging domain binds to CD16 and/or the NK activating domain comprises IL-15 or a functional fragment thereof. A nucleic acid molecule comprising a nucleotide sequence encoding the multispecific construct, a host cell comprising the nucleic acid, a method of making the multispecific construct, and a composition comprising the multispecific construct also are provided, as well as a method for stimulating expansion of NK cells and a method of treating cancer in a subject, the methods comprising administering to a subject in need thereof the multispecific construct of the disclosure.

The disclosure further provides a chimeric antigen receptor (CAR) comprising an extracellular domain comprising the CD83 binding protein, a transmembrane domain, and an intracellular signaling domain. In various aspects, the transmembrane domain is a CD8 transmembrane domain fused to the CD83 binding protein via a CD8 hinge, and the intracellular signaling domain comprises a 4-1BB costimulatory domain and a CD3-zeta intracellular signaling domain.

An immune effector cell (e.g., a T cell, a B cell, a Natural Killer (NK) cell, a NKT cell, a monocyte, a dendritic cell, an innate lymphoid cell, a cytokine induced killer (CIK) cell, or a lymphokine activated killer (LAK) cell) comprising the CAR also is contemplated. A method of suppressing alloreactive donor cells in a subject receiving transplant donor cells, the method comprising administering to the subject an effective amount of the immune effector cell is provided. Also provided is a method of treating cancer in a subject, the method comprising administering to the subject an effective amount of the immune effector cell described herein.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. For example, “a multispecific construct” is understood to represent one or more multispecific constructs. As such, the terms “a” (or “an”), “one or more,” and “at least one” can be used interchangeably herein. The term “or” should be understood to encompass items in the alternative or together, unless context unambiguously requires otherwise.

It should also be understood that when describing a range of values, the disclosure contemplates individual values found within the range. For example, “an amount of from about 10⁴ to 10⁹ cells/kg” could be, but is not limited to, 10⁵ cells/kg, 10⁶ cells/kg, 10⁷ cells/kg, etc., and any value in between such values. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The entire document is intended to be related as a unified disclosure, and it should be understood that all combinations of features described herein (even if described in separate sections) are contemplated, even if the combination of features is not found together in the same sentence, or paragraph, or section of this document. Section headings are provided only for readability of the disclosure, and are not meant to be limiting. Also, only such limitations which are described herein as critical to the invention should be viewed as such; variations of the invention lacking limitations which have not been described herein as critical are intended as aspects of the invention.

### BRIEF DESCRIPTION OF THE DRAWINGS

FIG. 1 is an illustration of an exemplary CD83 nanobody-chimeric antigen receptor (CAR) construct. A CD83 nanobody construct was de novo synthesized (GenScript) and inserted into a third-generation lentivirus vector under the control of the EF1α promoter. The construct comprised a CD8a leader followed by the CD83 VHH domain, CD8 hinge, CD8 transmembrane (TM) domain, 4-1BB and CD3z signaling domains linked to an RQR8 selection marker by a T2A ribosomal skipping sequence. Lentiviral particles comprising the CD83 nanobody-CAR expression construct were generated through the transient transfection of plasmid into 293T2 virus producing cells (ATCC) in the presence of Lipofectamine 3000 (Invitrogen, Carlsbad, CA, USA), VSV-G and packaging plasmids (Addgene, Cambridge, MA, USA).

FIG. 2 is an illustration of a mechanism of action for an exemplary TriKE of the disclosure. The TriKE is composed of an anti-CD16 camelid/nanobody (VHH, “cam16”), a linker (flanking region), an IL-15 moiety, a linker, and anti-CD83 camelid/nanobody (VHH, “cam83”). The cam16 and cam83 arms mediate a cytolytic synapse between NK cells (via CD16) and tumor cells (via CD83) that results in lysis of the tumor cell (via Granzymes and perforin) by the NK cells. The IL-15 moiety in the TriKE mediates NK cell expansion, priming, and survival of the NK cell.

FIGS. 3A and 3B are bar graphs demonstrating that the CD83 TriKEs of the disclosure mediate NK cell activation against U937 (FIG. 3A) and HL60 cancer cells (FIG. 3B). U937 or HL60 cells were exposed, at a 2:1 effector to target ratio to media alone (NT), 30 nM IL-15 (NCI IL-15), 30 nM MOPC TriKE (control, TriKE that does not engage tumor antigens), or 30 nM CD83 TriKE (x-axis). The percentage increase in IFN gamma production by NK cells was measured as a surrogate for NK cell activation (y-axis). The anti-CD83 TriKE mediated a significant increase in NK cell activation against the cancer cells.

FIG. 4A is a graph showing binding of CD83 nanobodies of the disclosure to CD83. Candidate nanobodies were screened, and a subset underwent affinity maturation. The anti-CD83 nanobodies, C10 and H1, bound to CD83+ target cells (activated CD4 T cells) with similar affinity to a commercial flow cytometry full length antibody (BD Pharmingen, HB15e clone).

FIG. 4B is a line graph illustrating suppression of a human, alloreactive T cell proliferation mediated by CD83 nanobodies described herein in seven-day mixed leukocyte reactions (compared to PBS vehicle control). **P=0.01-0.001 and ****P<0.0001. Concentration of nanobodies is noted on the x-axis in nM; % proliferation of T cells is noted on the y-axis.

FIG. 5 is a line graph illustrating cytotoxic activity of CAR T cells of the disclosure against cancer cells. The ratio of CAR T cells and U937 leukemia cells is provided on the x-axis, while percent cytotoxicity is noted on the y-axis. Bulk T cells transduced with the anti-CD83 CAR comprising SEQ ID NOs: 1-3 showed strong cytotoxicity at all ratios, whereas untransduced T cells (UTD) had a negligible cytotoxic effect on the target cells.

FIGS. 6A and 6B are line graphs illustrating results from the IncuCyte Assay described in Example 2. The ratios of cells used in the studies are noted: T cells 4:U937 cells 1 (4:1), T cells 1:U937 cells 1 (1:1), T cells 1:U937 cells 4 (1:4), and U937 cells only (without T cells). The tumor burden as measured as percent of Nuclear (Nuc) Light Intensity/time 0 is provided on the y-axis, while time (hours) is noted on the x-axis. Serial imaging was performed at one-hour intervals for 72 hours. Bulk T cells transduced with CD83-CAR significantly reduced the tumor burden at all ratios, whereas untransduced T cells (UTD) had a negligible effect on tumor burden as measured by the % of Nuclear Light Intensity/time.

FIG. 7 is listing of amino acid sequences and nucleic acid sequences referenced in the application. Sequences are provided for two representative CD83 binding proteins (referenced as C10 and H1 in the figure). In SEQ ID NOs: 8, 9, 18, and 19, bolded regions correspond to CDR sequences. The boundaries of the CDR sequences were determined using Kabat numbering. In SEQ ID NOs: 9 and 19, the italicized region corresponds to a 6×His tag, and the underlined and italicized region corresponds to an HA tag. The disclosure contemplates sequences similar to SEQ ID NOs: 9 and 19 lacking the tags. SEQ ID NO: 46 corresponds to a CAR construct amino acid sequence, wherein (N-terminus to C-terminus) the italicized region corresponds to a CD8a region, the bold region corresponds to the CD83 binding portion, the underlined region corresponds to a CD8 hinge region, the bold and italicized region corresponds to a CD8 transmembrane region, the double underline region corresponds to a 4-1BB region, the bold and dashed underline region corresponds to a CD3-zeta region, the italicized and double underline region corresponds to a T2A region, and the remainder of the sequence corresponds to an RQR8 region. The disclosure contemplates sequences similar to SEQ ID NO: 46 lacking the T2A and RQR8 regions.

FIGS. 8A-8D are bar graphs demonstrating that the CD83 TriKEs of the disclosure mediate NK cell activation against THP1 cells. The percentage increase in IFN gamma production by NK cells was measured as a surrogate for NK cell activation (y-axis). The anti-CD83 TriKE mediated a significant increase in NK cell activation against the cancer cells.

FIG. 9 illustrates data generated in Example 2. Human T cells transduced to express the CAR comprising the CD83 binding protein described herein (CD83 nanoCART) were cultured with 231-LM2 breast cancer target cells at an effector:target ratio of 5:1 or 10:1 for 24 or 48 hours. Controls included untransduced T cells (UT^{nano}). Target cell lysis (percentage) is denoted on the y-axis. A representative experiment performed in triplicate is shown. ***P=0.0001-0.001, ****P<0.0001.

### DETAILED DESCRIPTION

The disclosure provides a CD83 binding protein. CD83 is expressed on myeloid and lymphoid malignancies. CD83 is also expressed on alloreactive T cells, as well as mature or activated myeloid cells. The CD83 binding proteins, multispecific constructs, and anti-CD83 chimeric antigen receptor (CAR) T cells comprising the CD83 binding protein sequences provided herein are useful in a variety of contexts. For example, the CD83 binding proteins, multispecific constructs, and anti-CD83 chimeric antigen receptor (CAR) T cells comprising the CD83 binding protein sequences provided herein are useful for killing cancer cells, such as CD83+ hematologic malignancies (including, but not limited to, acute myeloid leukemia, T or B cell acute lymphoblastic leukemia/lymphoma, diffuse large B cell lymphoma, mantle cell lymphoma, chronic lymphocytic leukemia, myeloma, and Hodgkin lymphoma) and CD83+ solid malignancies (including, but not limited to, breast cancer, melanoma, lung cancer, ovarian cancer, cervical cancer, and colorectal cancer). The CD83 binding proteins, multispecific constructs, and anti-CD83 chimeric antigen receptor (CAR) T cells comprising the CD83 binding protein sequences provided herein also may be useful for treating or preventing alloimmune or autoimmune conditions, such as (but not limited to) rheumatoid arthritis, systemic lupus erythematosus, multiple sclerosis, Sjogren's syndrome, cutaneous sclerosis, and ulcerative colitis. As an additional example of aspects of the disclosure, the CD83 binding proteins, multispecific constructs, and anti-CD83 chimeric antigen receptor (CAR) T cells comprising the CD83 binding protein sequences provided herein are useful for treating or preventing rejection after solid organ or vascularized composite allotransplantation (e.g., heart, lung, liver, kidney, face, upper extremity, or abdominal wall tissue or organ transplantation) or cell therapy rejection (including, e.g., banked, off-the-shelf, allogeneic cell product (such as chimeric antigen receptor T cell or regulatory T cell therapy)). In yet another aspect of the disclosure, the CD83 binding proteins, multispecific constructs, and anti-CD83 chimeric antigen receptor (CAR) T cells comprising the CD83 binding protein sequences provided herein may be useful for preventing or treating acute or chronic GvHD after allogeneic hematopoietic cell transplantation. Merely to illustrate an exemplary aspect of the disclosure, the CD83 CAR described herein could be combined with an accompanying CAR or chimeric endocrine receptor (CER) targeting a different antigen, such as CD5, CD7, CD19, CD20, CD21, CD22, CD33, CD79b, CD123, BCMA, CLEC12A, FSH, or HER2, which, in various instances, improves safety, target specificity, or cytotoxic efficacy of a therapeutic. Additionally, the disclosure contemplates use of, e.g., the anti-CD83 chimeric antigen receptor (CAR) T cells described herein to treat or prevent cytokine release syndrome. These and other aspects of the disclosure are described further below.

The CD83 binding protein of the disclosure comprises the amino acid sequences of SEQ ID NO: 1, SEQ ID NO: 23, and SEQ ID NO: 24. In various aspects, the CD83 binding protein is a nanobody, also known as a single domain antibody or VHH, comprising a heavy chain complementarity determining region (HCDR)-1 comprising SEQ ID NO: 1, an HCDR2 comprising SEQ ID NO: 23, and an HCDR3 comprising SEQ ID NO: 24. Nanobodies have the benefit of being small (15 kDa), exhibiting greater tissue infiltration and target binding compared to larger constructs, and surviving acidic environments (such as, for instance, the leukemia niche) better than full length human antibodies. SEQ ID NO: 23 comprises the amino acid sequence INX₁X₂GTTN, wherein X₁ is H or Y and X₂ is E or D. Thus, in various aspects, the CD83 binding protein comprises an HCDR2 comprising SEQ ID NO: 2 or SEQ ID NO: 12. SEQ ID NO: 24 comprises the amino acid sequence X₃ARSIFGNS, wherein X₃ is I or N. In various aspects, the CD83 binding protein comprises an HCDR3 comprising SEQ ID NO: 3 or SEQ ID NO: 13. The disclosure provides a CD83 binding protein comprising the amino acid sequences of SEQ ID NO: 1 for HCDR1, SEQ ID NO: 2 for HCDR2, and SEQ ID NO: 3 for HCDR3. Alternatively, the disclosure provides a CD83 binding protein comprising the amino acid sequences of SEQ ID NO: 11 for HCDR1, SEQ ID NO: 12 for HCDR2, and SEQ ID NO: 13 for HCDR3.

Generally, nanobody structure entails CDR sequences interspersed with framework region (FR) sequences. A general nanobody structure comprises FR1-CDR1-FR2-CDR2-FR3-CDR3-FR4, wherein FR1 to FR4 refer to framework regions 1 to 4, respectively. Optionally, the binding protein comprises an FR1 amino acid sequence of SEQ ID NO: 25 (QVQLVESGGGLVQAGGSLTLSCAAX₄, wherein X₄ is S or F), such as the amino acid sequence of SEQ ID NO: 4 or the amino acid sequence of SEQ ID NO: 14. In various aspects, the binding protein comprises an FR2 amino acid sequence of SEQ ID NO: 5. In various aspects, the CD83 binding protein comprises an FR3 amino acid sequence of SEQ ID NO: 26 (YKDX₅VKGRFAISRDNAKNTVSLQMNSLX₆PEDTAVYFC, wherein X₅ and X₆ is S or T), such as the amino acid sequence of SEQ ID NO: 6 or the amino acid sequence of SEQ ID NO: 16. Optionally, the binding protein comprises an FR4 amino acid sequence of SEQ ID NO: 7.

In exemplary aspects, the disclosure provides a CD83 binding protein comprising the amino acid sequence of SEQ ID NO: 8, such as a binding protein comprising all or part of the amino acid sequence of SEQ ID NO: 9. In alternative aspects, the disclosure provides a CD83 binding protein comprising the amino acid sequence of SEQ ID NO: 18, such as a binding protein comprising all or part of the amino acid sequence of SEQ ID NO: 19.

The binding protein of the disclosure binds CD83, preferably binding more frequently, more rapidly, with greater duration and/or with greater affinity to CD83 than it does with alternative targets (e.g., other cell surface proteins). For example, the CD83 binding protein (or a CD83-targeted agent comprising the CD83 binding protein) preferably binds CD83 with greater affinity, avidity, more readily, and/or with greater duration than it binds to other non-CD83 proteins. It is also understood that, for example, a CD83 binding protein (or CD83-targeted agent) which specifically or preferentially binds to a first target may or may not specifically or preferentially bind to a second target. In general, under designated assay conditions, the CD83 binding protein (or CD83-targeted agent comprising the binding protein) binds preferentially to a CD83 and does not bind in a significant amount to other components present in a test sample.

A variety of assay formats may be used to select or characterize a CD83 binding protein. For example, solid-phase ELISA immunoassay, immunoprecipitation, BIAcore™ (GE Healthcare, Piscataway, NJ), fluorescence-activated cell sorting (FACS), Octet™ (ForteBio, Inc., Menlo Park, CA) and Western blot analysis are among many assays that may be used to characterize binding to CD83. Typically, a specific or selective reaction will be at least twice background signal or noise and more typically more than 10 times background. Optionally, the CD83 binding protein (or a CD83-targeted agent) binds CD83 with a K_{D} (Equilibrium Dissociation Constant=Antibody Off Rate/Antibody On Rate) of less than about 500 nM, such as less than about 200 nM, less than about 150 nM, less than about 100 nM, less than about 50 nM, less than about 10 nM (such as between about 10 nM and about 5 nM, or less than about 5 nM (e.g., between about 5 nM and 1 about nM). For example, the CD83 binding protein may bind CD83 with a K_{D} of about 1.5 nM to about 3.5 nM.

Additional features of the disclosure are described below, including CD83-targeted agents which comprise the CD83 binding protein described herein, compositions comprising the binding protein and/or CD83-targeted agent, methods of making the constructs described herein, and methods of using the constructs described herein.

### Multispecific Constructs

The disclosure provides a CD83-targeted construct comprising the CD83 binding protein described herein conjugated or fused to moieties with therapeutic activity and/or other binding specificity. For example, the disclosure contemplates a CD83 binding protein conjugated or fused to another binding moiety (which binds a target other than CD83) to create a bispecific binding construct or fused to multiple different binding moieties that bind targets other than CD83 to form multispecific binding constructs. In this regard, provided herein is a multispecific construct comprising an NK cell engaging domain, an NK activating domain operably linked to the NK engaging domain, and a targeting domain comprising the CD83 binding of the disclosure operably linked to the NK activating domain and the NK engaging domain. Natural killer (NK) cells are cytotoxic lymphocytes of the innate immune system capable of immune surveillance. NK cells express CD16, an activation receptor that binds to the Fc portion of IgG antibodies and is involved in antibody-dependent cell-mediated cytotoxicity (ADCC). NK cells are regulated by IL-15, which can induce increased antigen-dependent cytotoxicity, lymphokine-activated killer activity, and/or mediate interferon (IFN), tumor-necrosis factor (TNF) and/or granulocyte-macrophage colony-stimulating factor (GM-CSF) responses.

The exemplary multispecific construct described herein, in various aspects, drives NK-cell-mediated killing of target cells (e.g., CD83+ cells) and triggers an NK cell self-sustaining signal. The trispecific killer engager described herein is also referred to as a “TriKE.” TriKE molecules generally include one (or more) targeting domains (here, including the CD83 binding protein), and one or more cytokine NK activating domains (e.g., IL-15, IL-12, IL-18, IL-21, or other NK cell enhancing cytokine, chemokine, and/or activating molecule), with each domain operably linked to the other domains. As used herein, the term “operably linked” refers to direct or indirect covalent linking. Thus, two domains that are operably linked may be directly covalently coupled to one another. Conversely, the two operably linked domains may be connected by mutual covalent linking to an intervening moiety (e.g., a flanking sequence or linker). Two domains may be considered operably linked if, for example, they are separated by the third domain, with or without one or more intervening flanking sequences. Exemplary BiKE (bispecific killer engager) and TriKE molecules or compounds are further described in International Patent Publication No. WO 2017062604, the disclosure of which is incorporated herein by reference in its entirety.

The NK engaging domain can include any moiety that binds to and/or activates an NK cell and/or any moiety that blocks inhibition of an NK cell. Exemplary NK cell engaging domains include a moiety that binds to, e.g., CD16, CD16+CD2, CD16+DNAM, or CD16+NKp46. In some aspects, the NK engaging domain includes a moiety that binds to CD16. In some embodiments, the NK engaging domain activates an NK cell. In some embodiments, the NK engaging domain blocks inhibition of an NK cell.

In some aspects, the NK engaging domain selectively binds to a receptor at least partially located at the surface of an NK cell. In certain aspects, the NK engaging domain can serve a function of binding an NK cell and thereby bring the NK into spatial proximity with a CD83-positive cell. In certain embodiments, however, the NK engaging domain can selectively bind to a receptor that activates the NK cell and, therefore, also possess an activating function. Activation of the CD16 receptor can elicit antibody-dependent cell-mediated cytotoxicity. Thus, in certain aspects, the NK engaging domain includes a CD16 binding protein (e.g., at least a portion of an anti-CD16 receptor antibody or an antibody-like construct (scFv or nanobody) comprising CD16 binding affinity) effective to selectively bind to the CD16 receptor. In other aspects, the NK engager cell domain may interrupt mechanisms that inhibit NK cells. In such aspects, the NK engager domain can include, for example, anti-PD1/PDL1, anti-NKG2A, anti-TIGIT, anti-killer-immunoglobulin receptor (KIR), and/or any other inhibition blocking domain. The NK engaging domain can include an antibody (or fragment or antibody-like construct) or ligand that binds to any NK cell receptor such as, for example, the cell cytotoxicity receptor 2B4, low affinity Fc receptor CD16, killer immunoglobulin like receptors (KIR), CD2, NKG2A, TIGIT, NKG2C, LIR-1, and/or DNAM-1.

In some embodiments, the NK engaging domain includes an antibody, an antigen-binding fragment thereof, a nanobody, or an antibody-like construct in which CDR sequences are arranged in a non-naturally occurring scaffold which positions to the CDRs to bind the target antigen. “Antibody” refers generally to an intact immunoglobulin. Suitable antigen-binding antibody binding fragments include, e.g., a F (ab) 2 or a Fab. Suitable antibody-like constructs include, e.g., scFvs (a heavy variable chain component and a light variable chain component joined by a linker). In some aspects, the NK engaging domain includes a nanobody. In some aspects, the NK cell engager is a humanized CD16 engager derived from an animal nanobody. A nanobody may be derived from an antibody of any suitable animal such as, for example, a camelid (e.g., a llama or camel) or a cartilaginous fish. As described above, a nanobody can provide superior physical stability, an ability to bind deep grooves, and increased production yields compared to larger antibody fragments.

In one exemplary embodiment, a nanobody-based NK engager molecule comprises a humanized CD16 nanobody derived from a published llama nanobody (GeneBank sequence EF561291; Behar et al., 2008. Protein Eng Des Sel. 21(1):1-10), termed EF91. Llama EF91 showed functionality similar to rituximab-mediated killing in a chromium release assay with Raji targets. The CDRs were cloned into a humanized camelid scaffold (Vincke et al., 2009. J Biol Chem. 284(5):3273-3284) to humanize the CD16 engager, termed HuEF91. The binding of HuEF91 is equivalent to binding observed using a standard CD16 scFv, indicating that incorporating the llama nanobody variable heavy chain into the humanized backbone has not hindered the specificity of the molecule. The use HuEF91 as an NK engager in the TriKE molecules described herein can increase drug yield, increase stability, and/or increase NK-cell-mediated ADCC efficacy. In various aspects, the multispecific construct comprises an NK engager molecule comprising CDR sequence set forth in SEQ ID NOs: 28-30. NK engagers (and TriKEs generally) are further described in U.S. Patent Publication No. 20210388093, incorporated herein by reference in its entirety.

The NK activating domain of the multispecific construct can include an amino acid sequence that activates NK cells, promotes sustaining NK cells, or otherwise promotes NK cell activity. The NK activating domain can be, or can be derived from, one or more cytokines that activate and/or sustain NK cells. The term “derived from” refers to, e.g., an amino acid fragment of a cytokine (e.g., IL-15) that is sufficient to provide NK cell activating and/or sustaining activity. In aspects of the disclosure that include more than one NK activating domain, the NK activating domains may be provided in series or in any other combination. Additionally, each cytokine-based NK activating domain can include either the full amino acid sequence of the cytokine or may be an amino acid fragment, independent of the nature of other NK activating domains included in the TriKE molecule. Exemplary cytokines on which an NK activating domain may be based include, for example, IL-15, IL-18, IL-12, and IL-21. It will be appreciated that reference to an NK activating domain by identifying the cytokine on which it is based is applicable to the full amino acid sequence of the cytokine, any suitable amino acid fragment of the cytokine, and modified versions of the cytokine (e.g., including one or more amino acid substitutions (such as IL-15N72D or IL-15N72A)).

In some aspects, the NK activating domain comprises IL-15 or a functional fragment or variant thereof. In some aspects, the IL-15 is wild-type IL-15, preferably human IL-15. In some embodiments, the IL-15 comprises an amino acid sequence of SEQ ID NO: 36 or a functional variant thereof (e.g., a functional variant of IL-15 comprising an N72D or N72A amino acid substitution as compared to SEQ ID NO: 36). The use of IL-15 can provide sustained NK cell activity and/or can facilitate refolding of the construct and/or allow recovery in greater yield, thus rendering the TriKE molecules more suitable for clinical scale-up.

The term “functional variant” refers to a molecule, including a binding molecule, for example, comprising a nucleotide and/or amino acid sequence that is altered by one or more nucleotides and/or amino acids compared to the nucleotide and/or amino acid sequences of the parent molecule. For a binding molecule, a functional variant is still capable of competing for binding to the binding partner with the parent binding molecule. In other words, the modifications in the amino acid and/or nucleotide sequence of the parent binding molecule do not significantly affect or alter the binding characteristics of the binding molecule encoded by the nucleotide sequence or containing the amino acid sequence, i.e., the binding molecule is still able to recognize and bind its target. The functional variant may have conservative sequence modifications including nucleotide and amino acid substitutions, as well as additions and/or deletions (generally substitutions). Functional variants can also include, but are not limited to, derivatives that are substantially similar in primary structural sequence, but which contain chemical and/or biochemical modifications that are not found in the parent binding molecule. Such modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI-anchor formation, hydroxylation, iodination, methylation, myristoylation, oxidation, pegylation, proteolytic processing, phosphorylation, prenylation, racemization, selenoylation, sulfation, transfer-RNA-mediated addition of amino acids such as arginylation, ubiquitination, and the like.

In some embodiments, the multispecific construct further comprises a flanking sequence or linker sequence that links two of the above-described domains. The terms “flanking sequence” and “linker sequence” can be used interchangeably, unless context clearly indicates otherwise. In some aspects, the presence of the flanking sequence can further increase NK cell activation. Any amino acid sequence can be a flanking sequence or a linker sequence. One exemplary flanking sequence includes SEQ ID NO: 35. Another exemplary flanking sequence comprises SEQ ID NO: 37. Other exemplary linker sequences are set forth in SEQ ID NOs: 43-45. As yet another example, any number of repeats of an amino acid sequence can be a flanking sequence or a linker sequence. For example, any number of repeats of the sequence of SEQ ID NO: 45 can be a flanking sequence or a linker sequence. Sequence repeats can be complete or partial, and complete or partial repeats can be at the beginning, i.e., at the N-terminus, or the end, i.e., at the C-terminus, of a flanking sequence or linker sequence. Flanking sequences can be in any orientation. Flanking or linker sequences are further described in, e.g., U.S. Patent Publication No. 20210388093, incorporated herein by reference in its entirety, particularly with respect to the sequence listing.

The multispecific construct, in various instances, includes more than one flanking sequence. The flanking sequences that link the domains of the molecule can be the same or can be different. As an example, the same or different flanking sequences can link the NK engaging domain (e.g., CD16 nanobody) with the NK activating domain (e.g., IL-15) and the NK activating domain with the targeting domain comprising the CD83 binding protein. In some embodiments, the multispecific construct described herein includes at least one flanking sequence linking two of the domains, and optionally includes a second flanking sequence linking the two linked domains with a third domain. In some embodiments, the flanking sequences are the same. In some embodiments, the flanking sequences are different. In some aspects, the flanking sequences flank the NK activating domain.

A representative multispecific construct of the disclosure comprises a CD16 nanobody (NK engaging domain), a flanking sequence, IL-15 or fragment or variant thereof (NK activation domain), a flanking sequence, and a CD83 nanobody (targeting domain). In this regard, the disclosure provides a multispecific constructing comprising the amino acid sequence of SEQ ID NO: 39.

### Chimeric Antigen Receptors

The disclosure further provides a chimeric antigen receptor (CAR) polypeptide comprising the CD83 binding protein described herein. “Chimeric antigen receptor” or “CAR” refers to an artificial immune cell receptor that is engineered to recognize and bind to an antigen expressed by a target cell, such as a tumor cell. The CAR of the disclosure comprises an extracellular domain comprising the CD83 binding polypeptide described herein, a transmembrane domain, and an intracellular domain. Generally, a CAR is designed for a T cell and is a chimera of a signaling domain of the T cell receptor (TCR) complex and an antigen-recognizing domain (e.g., a single chain fragment (scFv) of an antibody or other antibody fragment) (Enblad et al., Human Gene Therapy. 2015; 26 (8): 498-505). CARs have the ability to redirect immune cell specificity and reactivity toward a selected target in a non-MHC-restricted manner. The non-MHC-restricted antigen recognition gives, e.g., T cells expressing CARs the ability to recognize an antigen independent of antigen processing, thus bypassing a major mechanism of tumor escape. Moreover, when expressed in T cells, CARs advantageously do not dimerize with endogenous T-cell receptor (TCR) alpha and beta chains.

There are various formats of CARs, each of which contains different components. “First generation” CARs join an antibody-derived scFv to the CD3-zeta intracellular signaling domain of the immune cell receptor through hinge and transmembrane domains. A cytoplasmic or intracellular signaling domain produces stimulatory signals for proliferation and effector function when the CAR engages with a target antigen. Many intracellular signaling domains contain signaling motifs which are known as immunoreceptor tyrosine-based activation motifs (ITAMs). Examples of ITAM containing cytoplasmic signaling sequences include those derived from CD8, CD3-zeta, CD3 gamma, CD3 epsilon, CD32 (Fc gamma RIIa), DAP10, DAP12, CD79a, CD79b, FcγRIγ, FcγRIIIγ, FcεRIβ (FCERIB), and FcεRIγ (FCERIG). Intracellular signaling domains, including those derived from CD3-zeta (CD3), are known in the art (TCR zeta, e.g., GenBank Accession No. BAG36664.1). “Second generation” CARs incorporate an additional domain, e.g., CD28, 4-1BB (41BB), or ICOS, to supply a costimulatory signal. In various aspects, the CAR comprises a costimulatory domain selected from CD27, 4-1BB, CD28, CD134, ICOS, OX40, CD149, DAP10, CD30, IL2-R, IL7r6, IL21-R, NKp30, NKp44, CD40, CD137, PD-1, ICOS, lymphocyte function-associated antigen-1 (LFA-1), CD2, CD7, LIGHT, NKG2C, B7-H3, or DNAM-1 costimulatory domains (or any combination thereof). Constructs comprising two, three, or more costimulatory domains are also envisioned. Costimulatory domains are further described in, e.g., U.S. Pat. No. 11,235,977, which is hereby incorporated by reference in its entirety and particularly with respect to the disclosure of CAR components. “Third generation” CARs contain two costimulatory domains (e.g., 4-1BB and CD28 costimulatory domains) fused with, e.g., the TCR CD3-zeta chain. Third generation costimulatory domains may include, e.g., a combination of CD3-zeta, CD27, CD28, 4-1BB, ICOS, or OX40. CARs, in some embodiments, contain an ectodomain (e.g., CD3-zeta), commonly derived from an antigen-binding domain (e.g., scFv), a hinge, a transmembrane domain, and an endodomain with one (first generation), two (second generation), or three (third generation) signaling domains derived from CD3 and/or co-stimulatory molecules. See, e.g., Maude et al., Blood. 2015; 125(26):4017-4023; Kakarla and Gottschalk, Cancer J. 2014; 20 (2): 151-155. CARs are further described in, e.g., U.S. Patent Publication No. 20200108098, hereby incorporated by reference in its entirety and particularly with respect to the disclosure of combinations of subunits of CAR constructs (see, e.g., Table 1 of U.S. Patent Publication No. 20200108098).

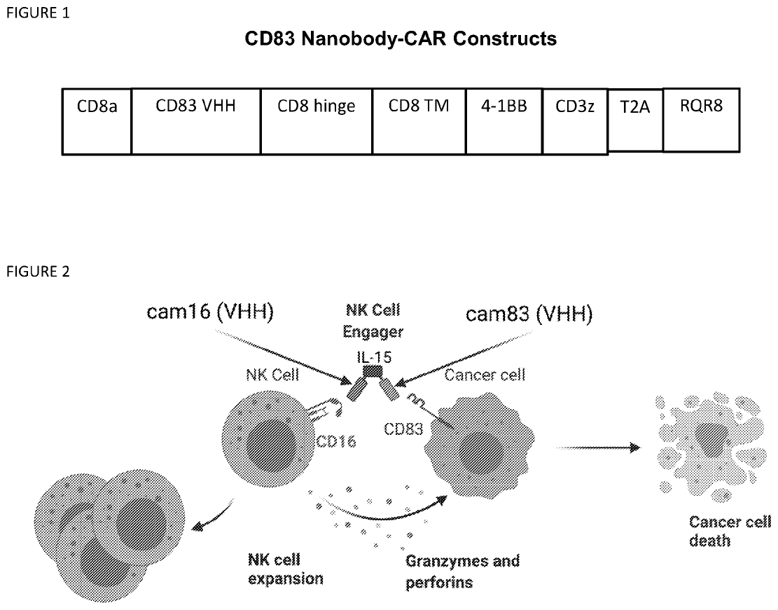

In various aspects, the CAR comprises a hinge domain which connects the CD83 binding domain to the transmembrane domain. Exemplary hinge domains include, but are not limited to, a CD28 hinge, a CD8 alpha hinge, a human IgG4 hinge domain, and a human IgG4 hinge domain combined with a CH3 human IgG4 domain. Hinge domains are further described in, e.g., Hudecek et al. (2013) Clin. Cancer Res., 19:3153, International Patent Publication No. WO 2014031687, and U.S. Pat. No. 8,822,647. In various aspects, the CAR comprises a CD8 hinge. Any suitable transmembrane domain may be used in the context of the CAR of the disclosure. Examples of transmembrane domains include, but are not limited to, domains comprising derived from CD28, CD3-epsilon, CD4, CD8, CD9, CD16, CD22, CD33, CD37, CD45, CD64, CD80, CD86, CD134, CD137, CD154, TCR-alpha, TCR-beta, H2-Kb, GITR, or CD3-zeta. In various aspects, the transmembrane domain is selected from CD3-zeta, CD28, or CD8 transmembrane domains. An exemplary CAR construct is illustrated in FIG. 1 and comprises the CD83 binding domain disclosed herein, a CD8 hinge, a CD8 transmembrane domain, a 4-1BB costimulatory domain, and a CD3-zeta intracellular signaling domain. In various aspects, the CAR is encoded by the nucleic acid sequence of SEQ ID NO: 27.

Immune effector cells genetically modified to express the CAR also are provided. These cells are optionally obtained from the subject to be treated (i.e., are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

The immune effector cells may comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. Indeed, examples of suitable immune effector cells include, but are not limited to Natural Killer (NK) cells, T cells (including cytotoxic T cells and/or regulatory T cells), monocytes, dendritic cells, and innate lymphoid cells (types 1-3). B cells, cytokine induced killer (CIK) cells, and a lymphokine activated killer (LAK) cells also are contemplated.

In various aspects, the immune effector cell is a T cell. T cells or T lymphocytes can be distinguished from other lymphocytes, such as B cells and natural killer cells (NK cells), by the presence of a T-cell receptor (TCR) on the cell surface. T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH7, TH9, or TFH, which secrete different cytokines to facilitate a different type of immune response.

Cytotoxic T cells (Tc cells or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, dampen T cell-mediated immunity toward the end of an immune reaction and to suppress auto-reactive T cells that escaped the process of negative selection in the thymus. Two major classes of CD4+ Treg cells have been described—naturally occurring Treg cells and adaptive Treg cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

The T cells may comprise a mixture of CD4+ cells. In other embodiments, the T cells are enriched for one or more subsets based on cell surface expression. In some instances, the T cells are cytotoxic CD8+ T lymphocytes. In some embodiments, the T cells comprise gamma-delta T cells, which possess a distinct T-cell receptor (TCR) having one gamma chain and one delta chain instead of alpha and beta chains.

Natural-killer (NK) cells are CD56+CD3− large granular lymphocytes that kill virally infected and transformed cells and constitute a critical cellular subset of the innate immune system (Godfrey J, et al. Leuk Lymphoma 2012 53:1666-1676). Unlike cytotoxic CD8+ T lymphocytes, NK cells launch cytotoxicity without the requirement for prior sensitization, and can also eradicate MHC-I-negative cells (Nami-Mancinelli et al. Int Immunol 2011 23:427-431).

Innate lymphoid cells are tissue-resident innate lymphocytes that produce particular cytokines in response to infection, inflammation, and tissue injury. Type 1 innate lymphoid cells (ILC1s) produce interferon (IFN) gamma. Type 2 innate lymphoid cells (ILC2s) express CRTH2, KLRG1, SST2, CD161, and CD25, and produce amphiregulin, and type 2 cytokines (e.g., IL-4, IL-5, and IL-13). Type 3 innate lymphoid cells (ILC3s) produce IL-22 as well as IL-17, IL-22, IFN-g, and GM-CSF, and can display NKp44, NKp30, and/or CD56 on the cell surface.

### Nucleic Acids, Host Cells

The disclosure further provides nucleic acid molecules comprising nucleic acid sequences encoding the CD83 binding protein described herein, as well as nucleic acid molecules encoding the multispecific construct and nucleic acid molecules encoding CARs. The nucleic acid molecule may be provided in an expression construct or vector, e.g., a plasmid, cosmid, YAC, or a viral vector. Expression vectors provide for expression in vitro and/or in vivo (e.g., in a suitable host cell or organism). Many expression vectors are commercially available. Suitable viral vectors include, for example, retrovirus, adenovirus, parvovirus (for example, adeno-associated viruses), coronavirus, negative strand RNA viruses such as ortho-myxovirus (for example, influenza virus), rhabdovirus (for example, rabies and vesicular stomatitis virus), paramyxovirus (for example, measles and Sendai), picornavirus, alphavirus, herpesvirus (for example, Herpes Simplex virus types 1 and 2, Epstein-Barr virus, cytomegalovirus), lentivirus, and poxvirus (for example, vaccinia, fowlpox, and canarypox). Vector components may include an origin of replication, one or more marker genes, a multiple cloning site containing recognition sequences for restriction endonucleases, enhancer elements, promoters, transcription termination sequences, and the like. Indeed, the nucleic acid of the disclosure may be operably linked to one or more regulatory elements, such as a promoter, enhancer, and/or terminator.

The disclosure further provides a host cell comprising the nucleic acid or the expression vector described herein. A “host cell” refers to a cell (e.g., prokaryotic or eukaryotic) into which exogenous nucleic acid has been introduced, including the progeny of such cells. A host cell may be a bacterial cell, a yeast cell, an insect cell, or a mammalian cell. In various aspects, the cell is a eukaryotic cell, such as a mammalian cell (e.g., a human cell; or cell from a non-human primate such as ape, chimpanzee, monkey, or orangutan; or cell from a domesticated animal, such as a dog or cat; or cell from livestock, such as a horse, cow, pig, sheep, or goat; or cell from another mammalian species including, without limitation, mice, rats, guinea pigs, rabbits, hamsters, birds (e.g., chicken, duck, goose, quail or pheasant) and the like). Examples of human cells include, but are not limited to, PER.C6 cells (described in, e.g., International Patent Publication No. WO 01/38362), MRC-5 (ATCC CCL-171), WI-38 (ATCC CCL-75), HEK-293 cells (ATCC CRL-1573), Hela cells (ATCC CCL2), and fetal rhesus lung cells (ATCC CL-160). Examples of non-human primate cells are Vero cells (ATCC CCL81), COS-1 cells (ATCC CRL-1650), and COS-7 cells (ATCC CRL-1651). Examples of dog cells are MDCK cells (ATCC CCL-34). Examples of rodent cells are hamster cells, such as BHK21-F, HKCC cells, or Chinese hamster ovary (CHO) cells. Examples of insect cells include, but are not limited to, SF9 cells (ATCC CRL-1711), Sf21 cells (IPLB-Sf21), MG1 cells (BTI-TN-MG1), and High Five™ cells (BTI-TN-5B1-4).

A method of making the CD83 binding protein described herein (as well as the multispecific construct described herein and the CAR described herein) is provided. The method comprises culturing a host cell comprising a nucleic acid encoding the CD83 binding protein, multispecific construct described herein, or the CAR described herein under conditions which allow expression, and recovering the product (i.e., the CD83 binding protein, multispecific construct, or the CAR). Culture conditions and methods for generating recombinant proteins are known in the art. Similarly, protein purification methods are known in the art and utilized herein for recovery of recombinant proteins from cell culture media. In some aspects, methods for protein purification include filtration, affinity column chromatography, cation exchange chromatography, anion exchange chromatography, and concentration. Optionally, the method comprises formulating the recovered product.

### Compositions, Methods of Use

Merely for convenience, the term “CD83-targeted agent” is used herein to reference any of the biological agents described herein comprising the CD83 binding protein comprising SEQ ID NOs: 1, 23, and 24, further described above. As such, the disclosure referencing a CD83-targeted agent may apply to the CD83 binding proteins described herein, the multispecific constructs, the CAR constructs, as well as immune effector cells displaying CARs comprising SEQ ID NOs: 1, 23, and 24. Thus, any of the disclosure herein in connection with “CD83-targeted agent” applies to any agent comprising a CD83 binding protein comprising the amino acid sequences of SEQ ID NO: 1 for HCDR1, SEQ ID NO: 2 for HCDR2, and SEQ ID NO: 3 for HCDR3; a CD83 binding protein comprising the amino acid sequences of SEQ ID NO: 11 for HCDR1, SEQ ID NO: 12 for HCDR2, and SEQ ID NO: 13 for HCDR3; a binding protein comprising an FR1 amino acid sequence of SEQ ID NO: 25, such as the amino acid sequence of SEQ ID NO: 4 or the amino acid sequence of SEQ ID NO: 14; a binding protein comprising an FR2 amino acid sequence of SEQ ID NO: 5; a binding protein comprises an FR3 amino acid sequence of SEQ ID NO: 26, such as the amino acid sequence of SEQ ID NO: 6 or the amino acid sequence of SEQ ID NO: 16; a binding protein comprising an FR4 amino acid sequence of SEQ ID NO: 7; a binding protein comprising the amino acid sequence of SEQ ID NO: 8, such as a binding protein comprising all or part of the amino acid sequence of SEQ ID NO: 9; and a binding protein comprising the amino acid sequence of SEQ ID NO: 18, such as a binding protein comprising all or part the amino acid sequence of SEQ ID NO: 19.

The disclosure provides a composition comprising any of the CD83-targeted agents described herein (e.g., the CD83 binding protein, the multispecific construct comprising the CD83 binding protein, or an immune cell comprising the CD83-targeted CAR) with a physiologically acceptable carrier. Physiologically acceptable carriers and excipients are preferably nontoxic to recipients under the administration conditions selected for a particular subject. Acceptable carriers and excipients include, for example, buffers (such as phosphate, citrate, HEPES, and TAE), antioxidants (such as ascorbic acid and methionine), preservatives (such as hexamethonium chloride, octadecyldimethylbenzyl ammonium chloride, resorcinol, and benzalkonium chloride), proteins (such as human serum albumin), hydrophilic polymers (such as polyvinylpyrrolidone), amino acids (such as glycine, glutamine, histidine, and lysine), and carbohydrates (such as glucose, mannose, sucrose, and sorbitol). Physiologically acceptable carriers also include, e.g., sterile water and physiological saline. Pharmaceutical/physiological compositions are preferably sterile and stable under conditions of manufacture and storage. Sterile solutions may be prepared, for example, by filtration through sterile filtration membranes.

The CD83-targeted agent of the present disclosure (e.g., the multispecific construct or the CD83-targeted CAR immune effector cell) may be administered by any acceptable route, including parenteral and subcutaneous routes of administration. Suitable routes include intravenous, intradermal, intramuscular, intraperitoneal, intranodal and intrasplenic, for example. In exemplary aspects, the therapeutic is provided via systemic (e.g., intravenous) administration.

The disclosure provides a method comprising administering to a subject in need thereof the multispecific construct comprising an NK cell engaging domain, an NK activating domain operably linked to the NK engaging domain, and a targeting domain comprising the CD83 binding protein operably linked to the NK activating domain and the NK engaging domain (TriKE). The TriKE is preferably administered in an amount effective to induce NK-mediated killing of a target cell. In various aspects, the target cell is a cancer cell.

The amount of CD83-targeted agent provided should be sufficient to achieve a desired biological effect in a clinically relevant period of time. An amount of multispecific construct of the disclosure may comprise, e.g., from about 100 ng/kg to about 50 mg/kg administered to the subject, including all integer values within those ranges, although in some embodiments the methods may be performed using a dose outside this range. In some aspects, the amount provided is about 0.01 mg/m² to about 10 mg/m². The amount of immune effector cell administered to a subject may comprise, e.g., a dose of 10⁴ to 10⁹ cells/kg body weight, such as 10⁵ to 10⁶ cells/kg body weight, including all integer values within those ranges. Multiple administrations are contemplated as needed to achieve a desired biological effect.

The disclosure also provides a method for stimulating expansion of NK cells, the method comprising administering to a subject in need thereof the multispecific construct described herein in an amount of effective to stimulate expansion of NK cells in the subject. NK cell expansion may be detected using routine laboratory techniques, such as cell count analysis, utilization of luminescent products to measure viable cell numbers (such as CellTiter-Glo), flow cytometric evaluation of dye (CFSE or CellTrace) dilution on NK cells (e.g., one week after treatment), or flow cytometric incorporation of Ki67 on proliferating cells (e.g., 5-7 days after treatment).

A method of treating cancer in a subject also is contemplated. The method comprises administering to the subject the multispecific construct described herein. Also provided is a method of treating cancer in a subject in need thereof, wherein the method comprises administering an immune effector cell displaying the CD83-targeted CAR described herein to the subject. In various aspects, the cancer is a cancer which expresses CD83, such as CD83+ hematologic malignancies and CD83+ solid malignancies (i.e. solid tumors). CD83 may be detected in a variety of ways. In various aspects, any method of the disclosure may comprise detecting CD83 expression in a target cell (e.g., cancer cell or immune cell). Methods of determining protein levels or identifying expression of proteins in a target cell type are well known in the art. Optionally, CD83 protein levels are determined via western blot or dot blot analysis, immunohistochemistry (IHC, e.g., quantitative immunohistochemistry), flow cytometry, immunocytochemistry, enzyme-linked immunosorbent assay (ELISA), enzyme-linked immunosorbent spot (ELISPOT; Coligan, J. E., et al., eds. (1995) Current Protocols in Immunology. Wiley, New York), radioimmunoassay, or chemiluminescent immunoassay. CD83 protein levels also may be measured via geometric mean fluorescence intensity (gMFI). CD83 may be quantified and monitored in real-time on cells of interest (e.g., cancer cells, CD4+ T cells, Tfh, or B cells) by, for example, peripheral blood draws and measured by flow cytometry. CD83 RNA is optionally measured via real time polymerase chain reaction (qRT-PCR) or RNA sequencing. The sequence of CD83 is known in the art. See, e.g., Genbank Accession Nos. NM_001040280 and NM_004233.

The cancer is optionally prostate cancer, lung cancer, colon cancer, rectum cancer, urinary bladder cancer, melanoma, kidney cancer, renal cancer, oral cavity cancer, pharynx cancer, pancreas cancer, uterine cancer, thyroid cancer, skin cancer, head and neck cancer, cervical cancer, ovarian cancer, or hematopoietic cancer. In various aspects, the cancer is a CD83+ hematologic malignancy, such as but not limited to, acute myeloid leukemia, T or B cell acute lymphoblastic leukemia/lymphoma, diffuse large B cell lymphoma, mantle cell lymphoma, chronic lymphocytic leukemia, myeloma, and Hodgkin lymphoma. In various aspects, the cancer is a CD83+ solid malignancy, such as but not limited to, breast cancer, melanoma, lung cancer, ovarian cancer, cervical cancer, and colorectal cancer. When appropriate, the method may comprise administering the multispecific construct (or other construct comprising the CD83 binding protein described herein) prior to, simultaneously with, or following, a different therapy, such as chemotherapy, surgical resection of a tumor, or radiation therapy.

In a representative aspect, the cancer is acute myeloid leukemia (AML). The CD83 targeted agents described herein have significant advantages compared to currently available CAR T cell therapeutics for AML. Existing anti-AML CAR (e.g., which target CD33 or CD123) involve a risk of myeloid aplasia and often require follow-up allogeneic hematopoietic cell transplantation a few weeks after cell infusion. In contrast, CD83 is significantly reduced or absent from healthy hematopoietic stem cells, myeloid precursors in the marrow, and steady state circulating neutrophils, and therefore is not associated with the risks of existing treatment options.

The disclosure also provides a method of treating or reducing the risk of developing an alloimmune or autoimmune condition in a subject in need thereof. The method comprises administering to the subject a CD83-targeted agent described herein, such as the CD83-targeted multispecific construct described herein or the immune effector cell displaying the CD83-targeted CAR described herein. Autoimmunity is an immune response to self-antigens, which results in the body attacking normal cells and tissues. There are more than 80 types of autoimmune diseases that present unique clinical symptoms and affect different parts of the body. In contrast, alloimmunity is an immune response to antigens from different members of the same species. Alloimmunity is triggered primarily by differences in the major histocompatibility complex between individuals within a species. The body's response to alloimmune conditions and autoimmune conditions mimics the response against infection, involving activation of immune cells, inflammation, and tissue damage. In various aspects, the alloimmune or autoimmune condition is Graft versus Host Disease (GvHD), systemic lupus erythematosus (SLE), multiple sclerosis, Sjogren's syndrome, systemic sclerosis/scleroderma, cutaneous sclerosis, ulcerative colitis, inflammatory bowel disease, or rheumatoid arthritis.

Graft versus Host Disease (GvHD) occurs when donor immune cells transferred to an allogeneic recipient attack tissues in the recipient. The skin, liver, and intestinal tract are often affected, resulting in significant damage and morbidity. GvHD is a common complication of allogeneic hematopoietic cell transplantation (HCT), but also may develop after solid organ transplantation. In various aspects, the alloimmune or autoimmune condition is GvHD. The GvHD may be acute GvHD or may be chronic GvHD. While the symptoms of acute and chronic GvHD overlap, acute and chronic GvHD are immunologically distinct complications (e.g., complications of alloHCT). Acute GvHD is primarily mediated by alloreactive T cells. Acute GvHD is potentiated by early tissue damage during the conditioning regimens (e.g., radiation and/or chemotherapy) used to prepare patients to receive allogeneic hematopoietic cells. Acute GvHD typically impacts the skin, gut, and liver and often occurs before day 100 post-transplant. Chronic GvHD stems from a unique triad of thymic impairment, auto- and alloantibody production by reactive B cells, and systemic tissue fibrosis. Chronic GvHD is a systemic disease affecting host mucocutaneous tissues, musculoskeletal system, gastrointestinal tract, liver, lungs, immune system, and cardiovascular system. Chronic GvHD typically occurs later after transplantation (e.g., after day 100 post alloHCT), and can essentially develop at any time, even decades after the procedure.

The disclosure provides a method for treating or preventing rejection after solid organ or vascularized composite allotransplantation (e.g., heart, lung, liver, kidney, face, upper extremity, or abdominal wall tissue or organ transplantation) or rejection associated with a cell therapy (including, e.g., banked, off-the-shelf, allogeneic cell therapy (such as chimeric antigen receptor T cell or regulatory T cell therapy)). In yet another aspect of the disclosure, the disclosure provides a method for preventing or treating acute or chronic GvHD after allogeneic hematopoietic cell transplantation. The method comprises administering to the subject in need thereof a CD83-targeted agent described herein, such as the CD83-targeted multispecific construct described herein or the immune effector cell displaying the CD83-targeted CAR described herein. In aspects of the method wherein the immune effector cell displaying the CD83-targeted CAR is employed in the method, the CD83 CAR could be combined with a different CAR or chimeric endocrine receptor (CER) targeting a different antigen, such as CD5, CD7, CD19, CD20, CD21, CD22, CD33, CD79b, CD123, BCMA, CLEC12A, FSH, or HER2.

Also provided is a method of suppressing alloreactive donor cells in a subject receiving transplant donor cells, the method comprising administering to the subject an effective amount of the immune effector cell displaying the CD83-targeted CAR construct described herein. Alternatively, the method of suppressing alloreactive donor cells in a subject receiving transplant donor cells comprises administering to the subject an effective amount of CD83-targeted multispecific construct described herein. In various aspects, the donor cells are bone marrow cells, such as bone marrow cells comprising alloreactive T-cells, dendritic cells, or a combination thereof. Optionally, the subject has not received an immunosuppressant (e.g., a calcineurin-inhibitor (such as cyclosporine A or tacrolimus), a glucocorticoid, cyclophosphamide, ruxolitinib, or methotrexate).

Further provided is a method of treating or inhibiting cytokine release syndrome (CRS) in a subject in need thereof. CRS is a complication associated with immunotherapies, such as conventional CAR T cell therapy, wherein patients may develop fever, circulatory collapse, arrhythmia, hypoxia, multiorgan failure, or death within days to weeks of treatment. CRS is often associated with abnormal IL-6 levels, which is produced by mature or activated myeloid cells, which often also express CD83 and, as such, is a target for the CD83 targeted agents described herein. In various aspects, the CD83 targeted agent is administered to the subject prior to receiving another immunotherapy, is administered to the subject concurrently with the immunotherapy, or is administered after the different immunotherapy treatment is provided to the subject.

The term “treat,” as well as words related thereto, does not necessarily imply 100% or complete treatment. Rather, there are varying degrees of treatment of which one of ordinary skill in the art recognizes as having a potential benefit or therapeutic effect. In this respect, the methods of treating a condition or disease of the present disclosure can provide any amount or any level of treatment. Furthermore, the treatment provided by the method may include treatment of one or more conditions or symptoms or signs of the disease being treated. For instance, the treatment method of the present disclosure may inhibit one or more symptoms of the disease. Also, the treatment provided by the methods of the present disclosure may encompass slowing the progression of the disease. The treatment provided by the presently disclosed method may delay the onset or reoccurrence/relapse of the disease being treated. For example, a therapeutic response would refer to one or more of the following improvements in a disease or condition: (1) a reduction in the number of neoplastic or unwanted immune cells; (2) an increase in target (e.g., neoplastic) cell death; (3) inhibition of target (e.g., neoplastic) cell survival; (4) inhibition (i.e., slowing to some extent, preferably halting) of tumor growth or appearance of new lesions in the context of cancer; (5) slowing of disease progression; (6) an increased patient survival rate; (7) downgrade of stage of a disease (e.g., Stage 2 to Stage 1) and/or (8) some relief from one or more symptoms associated with the disease or condition. Disease states may be monitored by, e.g., clinical examination, X-ray, computerized tomography (CT, such as spiral CT), magnetic resonance imaging (MRI), positron emission tomography (PET), ultrasound, endoscopy and laparoscopy, tumor marker levels in the context of cancer (e.g., carcinoembryonic antigen (CEA)), cytology, histology, biopsy sampling, and/or counting of target cells in circulation. These methods also are typically used to diagnose and stage, e.g., cancer. The term “effective,” in various aspects of the disclosure, refers to a sufficient quantity of a composition which ameliorates one or more causes or symptoms of a condition or disease.

In certain aspects, the method of treating a disease or condition may be regarded as a method of inhibiting the disease or condition or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the condition or a symptom thereof to any amount or level.

When appropriate, the method may comprise administering the CD83-targeted agent (e.g., multispecific construct or immune effector cell) prior to, simultaneously with, or following, a secondary treatment, such as immunotherapy, chemotherapy, surgical resection of a tumor, radiation therapy, and the like. In some embodiments, the method(s) described herein further comprises administration of one or more other therapeutic agents. In this regard, the method optionally comprises administering to the subject a second therapeutic agent selected from the group consisting of a corticosteroid, methotrexate, cyclosporine, mycophenolate mofetil, tacrolimus, sirolimus, everolimus, antithymocyte globulin, alemtuzumab, dexamethasone, cyclophosphamide, ibrutinib, imatinib, infliximab, etanercept, tocilizumab, alemtuzumab, basiliximab, daclizumab, rituximab, denileukin diftitox, pentostatin, ruxolitinib, belumosudil, abatacept, cyclosporine, thalidomide, bortezomib, lenalidomide, halofuginone, hydroxychloroquine, mesenchymal stem cells, type 2 innate lymphoid cells, and regulatory T cells (or any combination thereof, potentially in further combination with other therapeutics). In various aspects of the disclosure, the methods described herein further comprise administering chemotherapy to the subject; optionally the chemotherapy is altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, or vinorelbine. The CD83-targeted agents may be used in combination with radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytotoxin, fludarabine, rapamycin, mycophenolic acid, steroids, FR901228, and/or cytokines. In some embodiments, the CD83-targeted agents are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In various aspects, the CD83-targeted agent may be administered following B-cell ablative therapy such as agents that react with CD20, e.g., RITUXAN®, For example, in transplant situations, subjects may undergo standard treatment with high dose chemotherapy followed by peripheral blood stem cell transplantation. In certain embodiments, following the transplant, subjects receive an infusion of the expanded immune effector cells of the present disclosure. In an additional embodiment, immune effector cells are administered before or following surgery.

While the methods described herein are described as methods of treatment, it will be appreciated that the disclosure further contemplates use of any of the CD83-targeted agents in the treatment of any of the diseases, disorders, or conditions described herein. Use of any of the CD83-targeted agents in the manufacture of a medicament for the treatment of any of the diseases, disorders, or conditions described herein also is provided, as is the CD83-targeted agents for use in the treatment of any of the diseases, disorders, or conditions described herein.

The terms “subject in need” or those “in need of treatment” include subjects already afflicted with the disorder, as well as those in which the disorder is to be prevented. The “subject in need” or “patient” includes human and other mammalian subjects that receive either prophylactic or therapeutic treatment. The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). Preferably, the mammal is a human.

As an additional aspect, provided herein are kits which comprise a composition described herein packaged in a manner which facilitates its use for administration to subjects. In one embodiment, such a kit includes a composition described herein, packaged in a container such as a sealed bottle, vessel, single-use or multi-use vial, prefilled syringe, or prefilled injection device, optionally with a label affixed to the container or included in the package that describes use of the formulation in practicing the method. In one aspect, the formulation is packaged in a unit dosage form. The kit may further include a device suitable for administering the formulation according to a specific route of administration. Preferably, the kit contains a label that describes use of the formulation described herein.

The invention is further described in the following Examples. The Examples serve only to illustrate the invention and is not intended to limit the scope of the invention in any way.

### EXAMPLES

### Example 1

The Example describes the generation and characterization of a TriKE of the disclosure.

FIG. 2 is an illustration of an exemplary TriKE of the disclosure comprising an anti-CD16 camelid/nanobody (VHH, “cam16”), a linker, an IL-15 moiety, a linker, and anti-CD83 camelid/nanobody (VHH, “cam83”). The anti-CD83 nanobody comprised the amino acid sequence of SEQ ID NO: 18 (comprising the CDR sequences of SEQ ID NOs: 11-13). The anti-CD16 portion of the construct comprised the amino acid sequences of SEQ ID NOs: 28-30. The TriKE was cloned using Gibson Method cloning and expressed in Expi293 cells. After four days, the supernatant was harvested, and the TriKE was isolated using affinity chromatography via a 10×HIS-tag present at the C-terminal end of the protein. The schematic in FIG. 2 depicts a proposed mechanism of action of this molecule: the cam 16 and cam83 arms mediate a cytolytic synapse between NK cells (via CD16) and tumor cells (via CD83) that results in lysis of the tumor cell (via Granzymes and perforin) by the NK cells. The IL-15 moiety in the TriKE mediates NK cell expansion, priming and survival of the NK cell.

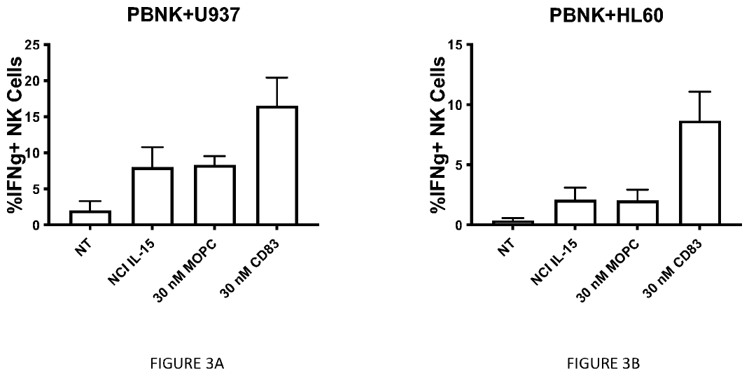

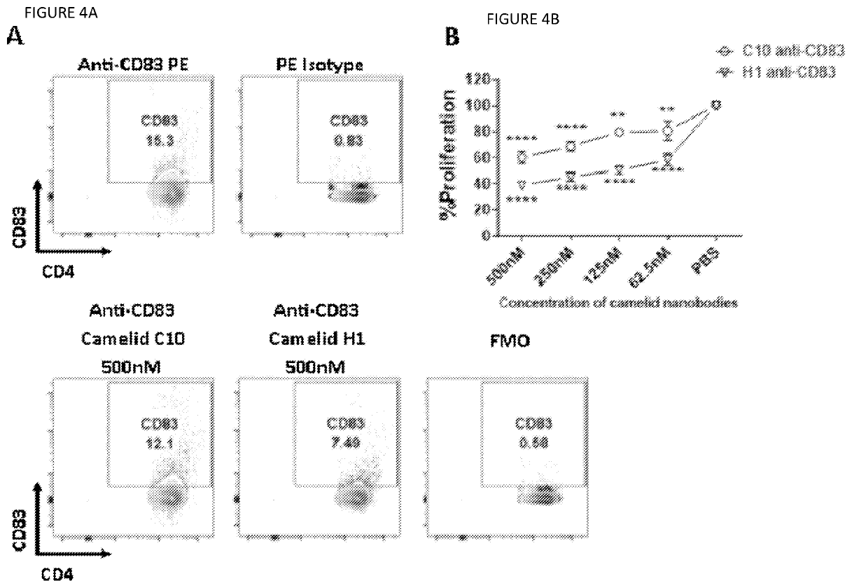

Peripheral blood mononuclear cells were incubated with U937 (pro-monocytic, human myeloid leukemia) or HL60 (pro-myelocytic leukemia) cells at a 2:1 effector to target ratio in the presence of media alone (NT), 30 nM IL-15 (NCI IL-15), 30 nM MOPC TriKE (control, TriKE that does not engage tumor antigens), or 30 nM CD83 TriKE for five hours. One hour into incubation, golgi plug/stop was added to prevent cytokine secretion. Cells were stained for surface antibodies to distinguish NK cells (CD56+CD3−), fixed, permeabilized, and intracellular antibodies against interferon gamma (IFNg) were used to evaluate the impact of the treatments on NK cell activation (IFNg production). N=3. The results of the assay are illustrated in FIGS. 3A and 3B. The CD83 TriKE, composed of an anti-CD16 camelid/nanobody, an IL-15 moiety, the CD83 binding protein of the disclosure induced superior function against both cell lines when compared to no treatment (NT), IL-15 alone, or IL-15 in the context of a TriKE that does not mediate a cytolytic synapse (MOPC) as the tumor binding arm is an isotype control that does not bind tumor.

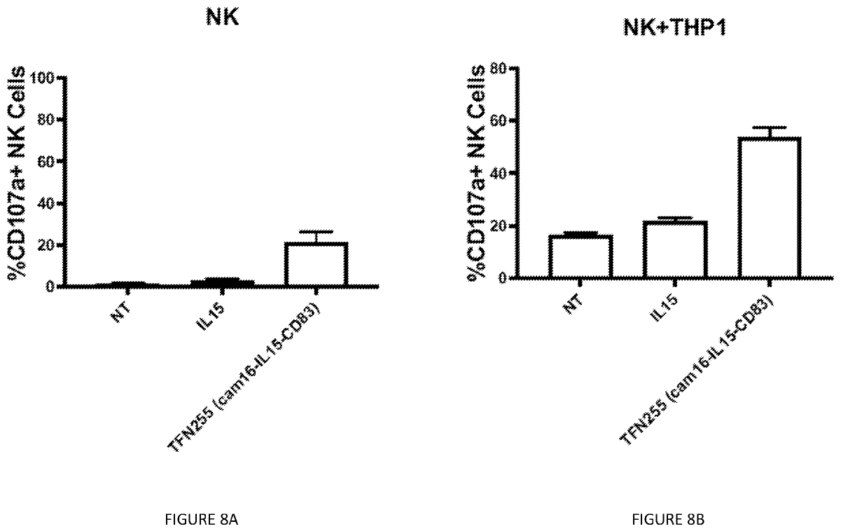

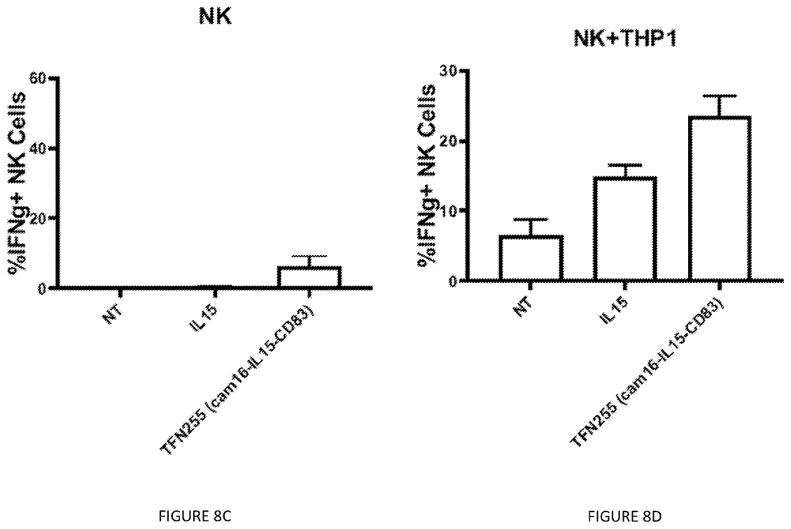

A similar study was performed with THP1 (acute monocytic leukemia) cells. The results are provided in FIGS. 8A-8D. The CD83 TriKE, composed of an anti-CD16 camelid/nanobody, an IL-15 moiety, the CD83 binding protein of the disclosure induced superior function against this additional cancer type when compared to no treatment (NT) and IL-15 alone.

This Example describes generation of a multispecific antigen binding protein comprising a CD83 antigen binding protein of the disclosure fused to a CD 16 antigen binding protein and further comprising IL-15. The TriKE retained binding activity and biologic activity of each moiety (IL-15, CD83 VHH, and CD16 VHH) of the construct. The construct exhibited activity against three different cancer cell types.

### Example 2

This Example describes the generation and characterization of a CAR T cell of the disclosure.

Peripheral blood mononuclear cells (PBMC) were isolated from de-identified normal donor blood apheresis cones, using SepMate tubes (STEMCELL Technologies, Vancouver, Canada). Bulk T cells were isolated from the PBMC with negative selection magnetic beads using EasySep™ Human T Cell Isolation Kit (STEMCELL Technologies, Vancouver, Canada). Primary cells were cultured in T Cell Medium made with X-Vivo 15 (Lonza, Walkersville, MD, USA) supplemented with 10% human serum albumin and Penicillin-Streptomycin-Glutamine. T cells isolated from healthy donors were stimulated with Cell Therapy Systems Dynabeads CD3/CD28 (Life Technologies) at a 1:3 cell to bead ratio. The cells were transduced with lentiviral particles comprising nucleic acid molecules encoding the chimeric antigen receptor construct illustrated in FIG. 1. The CD83 binding portion of the CAR comprised the sequence of SEQ ID NO: 8 (which comprises the HCDR sequences of SEQ ID NOs: 1-3). The coding sequence was placed under the control of an EF1α promoter. An RQR8 selection marker was included, linked to the coding sequence via a T2A ribosomal skipping sequence. Transduction was performed 24 hours after stimulation at a multiplicity of infection (MOI) of 3.0. CAR expression on T cells was determined by flow cytometry with fluorescent-conjugated antibodies directed at the RQR8 selection marker. Approximately 90% of the cells expressed the CAR on the cell surface. Magnetic bead removal was performed on day 7 and CAR T cells were harvested and used on Day 8 for experiments.

The ability of the CAR T cells to kill cancer cells and reduce tumor burden was evaluated. U937 cells (CD83+ Acute Myeloid Leukemia (AML) cell line) were transduced with a luciferase-GFP lentivirus and sorted to 100% purity. The cell line was cultured in R10 (made with RPMI 1640, 10% fetal bovine serum and 1% Penicillin-Streptomycin-Glutamine). For luciferin-based cytotoxicity assays, U937 cells were incubated with anti-CD83-CAR T cells comprising the CD83-binding peptide described herein at indicated ratios for 24, 48, or 72 hours as specified in each specific experiment. For the IncuCyte assay, U937 cells were incubated with the CAR T cells or untransduced (UTD) T cells at indicated ratios and serial images were obtained hourly for 72 hours.

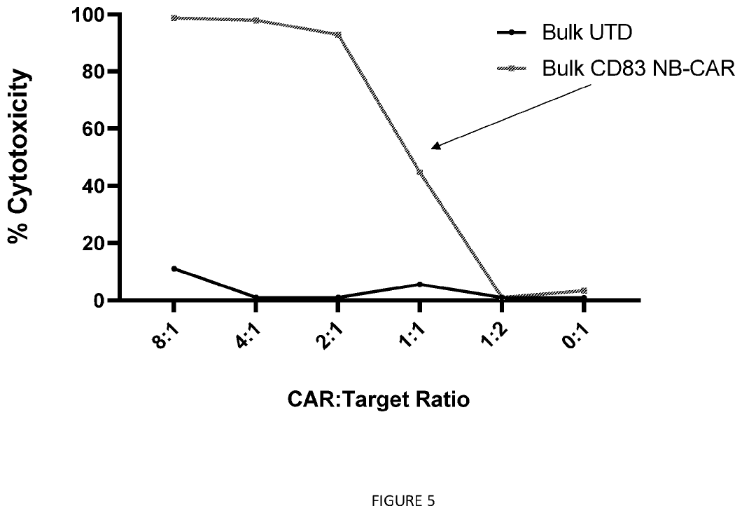

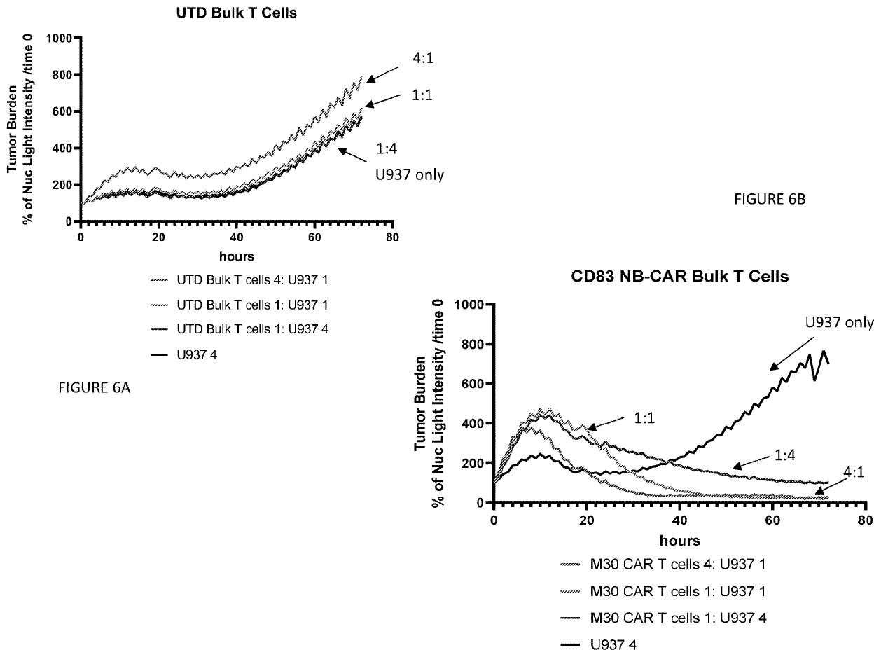

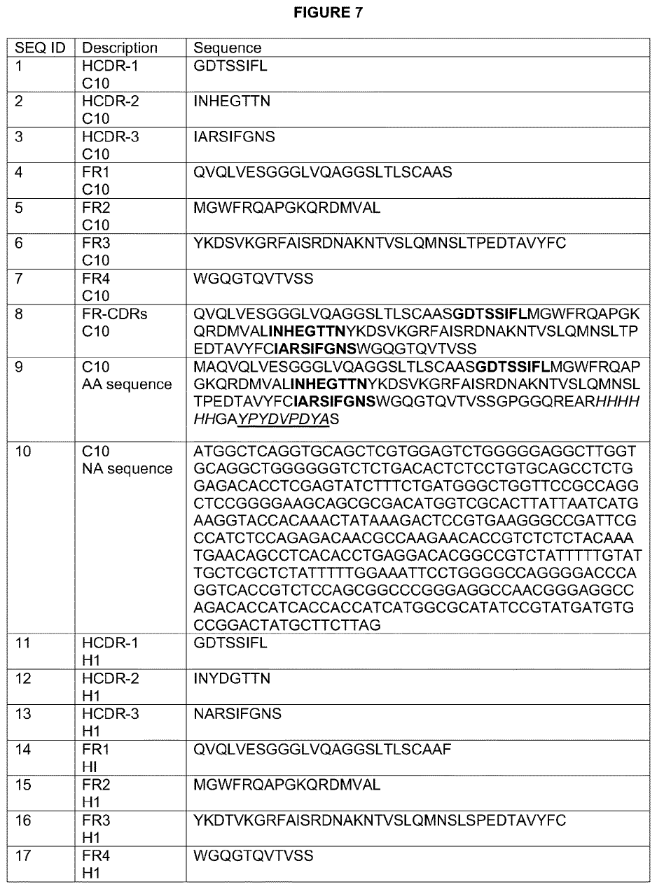

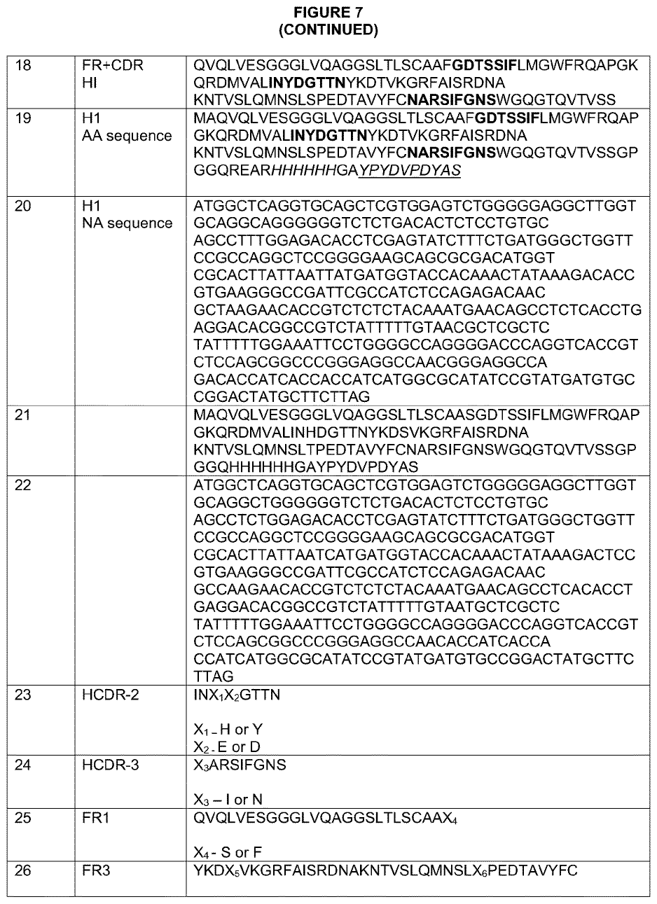

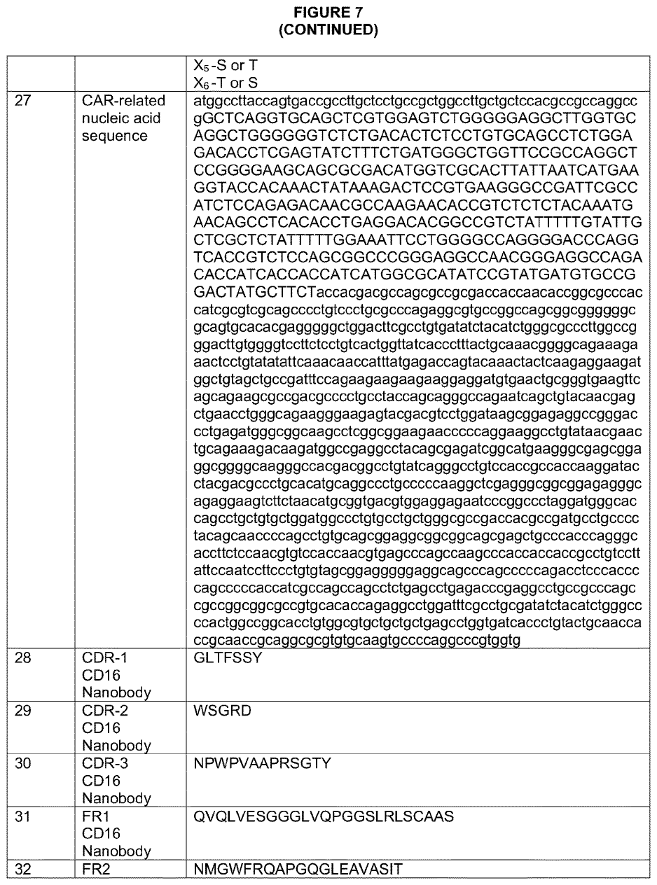

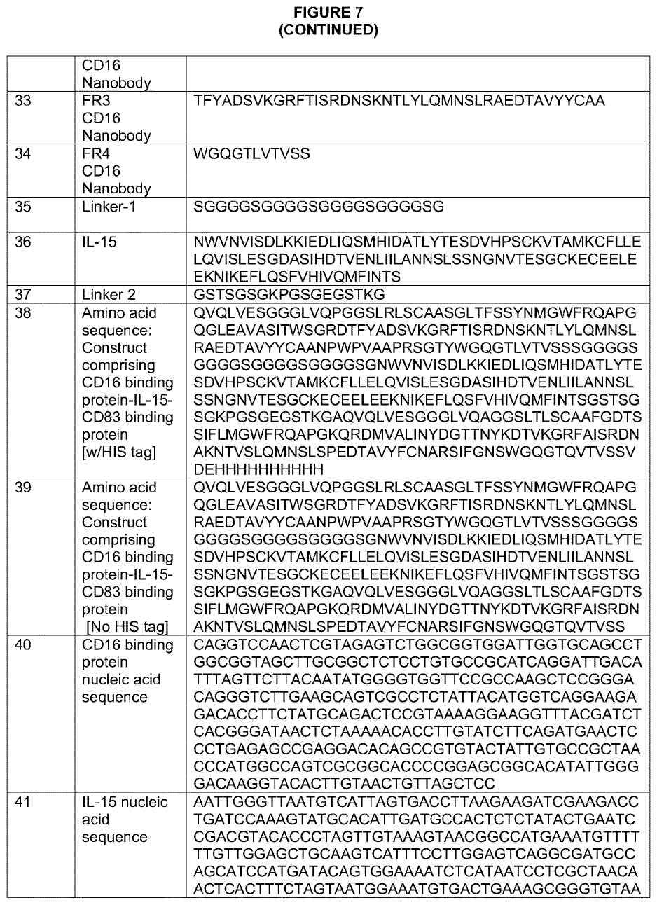

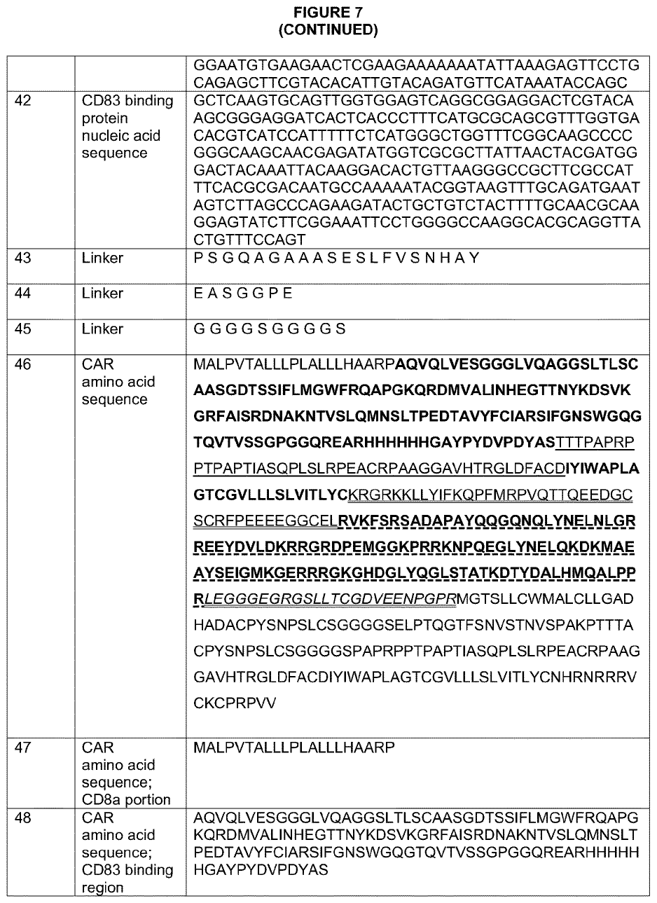

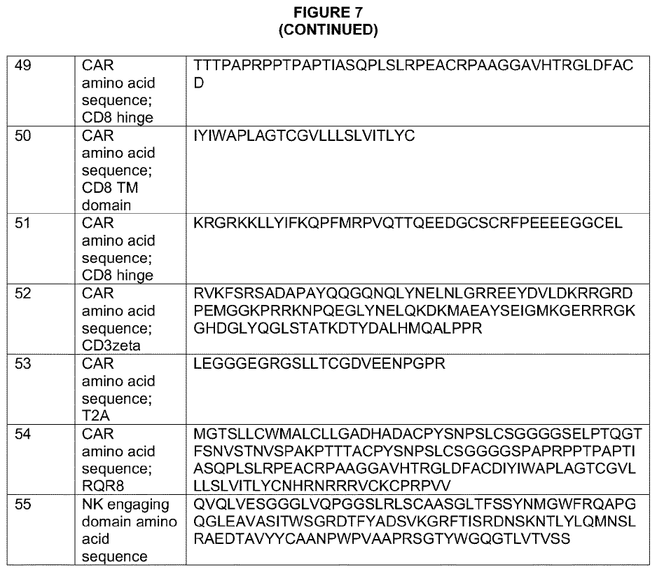

The results of the cytotoxicity assays are illustrated in FIGS. 5 and 6. FIG. 5 illustrates the results from the luciferin-based cytotoxicity assay wherein the CD83+ Luciferase+AML cell line U937 was incubated with the CAR T cells at indicated ratios for 48 hours. Bulk T cells transduced with the CD83-CAR showed strong cytotoxicity at all ratios, whereas untransduced T cells (UTD) demonstrated a negligible cytotoxic effect on the target cells. FIGS. 6A and 6B illustrate the results from the IncuCyte assay. Bulk T cells transduced with CD83-CAR significantly reduced the tumor burden at all ratios, whereas untransduced T cells had only a negligible effect on tumor burden as measured by the % of Nuclear Light Intensity/time.

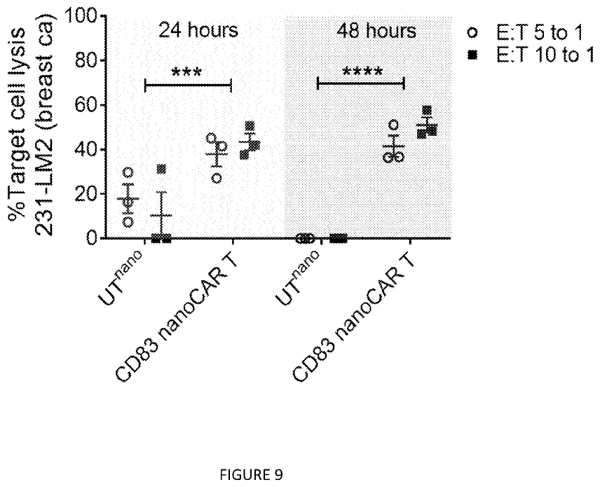

A cytotoxicity assay was also performed using 231-LM2 breast cancer cells. Human T cells transduced to express the CAR comprising the CD83 binding protein described herein (CD83 nanoCART) were cultured with 231-LM2 breast cancer target cells at an effector:target ratio of 5:1 or 10:1 for 24 or 48 hours. Controls included untransduced T cells (UT^{nano}). The results are illustrated in FIG. 9. The CAR T cells described herein mediated robust killing of 231-LM2 target cells. The study demonstrates the tumoricidal activity of the CAR T cell described herein against another CD83-expressing cancer (here, human breast cancer).

This Example describes the generation of CAR T cells comprising a CD83 binding protein of the disclosure (e.g., a CD83 binding protein comprising SEQ ID NOs: 1-3). The results summarized herein establish that the CD83 CAR T cells are effective in killing cancer cells expressing CD83 and reducing tumor burden.

### Example 3

This example describes methods for evaluating the activity of a CAR T cell of the disclosure.

Prevention of xenogeneic graft versus host disease. This model is clinically relevant as it uses human T cells that are activated against host antigen and host tissues. Target organs of this model include the lungs, skin, GI tract, and liver, replicating human pathology. NSG® mice will receive 10-30×10e6 human peripheral blood mononuclear cells (PBMC) on day 0 by i.p. injection to induce xenogeneic GVHD. Groups of mice will also receive 0.1-10×10e6 CAR T cells targeting CD83 or mock transduced T cell (controls) once within days 0-14 by i.p. injection. Mice will be followed twice a week for clinical GVHD (skin, fur, posture, mobility, and weight) up to day +80. Animals will be monitored and euthanized in cases of distress or if pre-moribund. The clinical endpoint is premoribund state. In cases of distress (including but not limited to hypoactivity, failure to respond to stimuli, vocalizations, shivering, ataxia, pale mucous membranes, labored breathing, and/or paresis, paralysis), loss of >20% body weight, or moribund state, animals will be immediately euthanized (CO₂ followed by cervical dislocation in accordance with AVMA guidelines). Kaplan Meier curves will be constructed to determine survival.

Treatment of human myeloid or lymphoid leukemia. This model is clinically relevant as it uses engineered human CAR T cells to eliminate systemic myeloid or lymphoid leukemia, closely replicating human disease. NSG® mice will receive 0.1-5×10e6 luciferase-transduced Raji (lymphoid leukemia) or MV411 (myeloid leukemia) or U937 (myeloid leukemia) cells by i.p. injection or tail vein injection. Groups of mice will also receive 0.1-10×10e6 CAR T cells targeting CD83 once within days 0-14 by i.p. injection. Controls groups will receive 0.1-10×10e6 mock transduced T cells once within days 0-14 by i.p. injection or leukemia alone. BLI assessments will be performed twice a week (luciferin 150 mg/kg) up to day +60. During these experiments using live tumor cells, animals will be euthanized (CO₂ followed by cervical dislocation in accordance with AVMA guidelines) in cases of distress (including but not limited to hypoactivity, failure to respond to stimuli, vocalizations, shivering, ataxia, pale mucous membranes, labored breathing, and/or paresis, paralysis); if tumor grows >15 mm, ulcerates, restricts locomotion, or causes >10% body weight gain; or if premoribund. Any visually observed tumors will be measured twice a week with calipers. The endpoints for these experiments will be tumor growth by BLI. Mice will be euthanized if a tumor endpoint is met (including strong BLI). Non-lethal, saphenous vein blood draws may be performed every 2 weeks (volume up to 1% mouse body weight) to enumerate CAR T cells and any circulating tumor cells.

All references, including publications, patent applications, and patents, cited herein are hereby incorporated by reference to the same extent as if each reference were individually and specifically indicated to be incorporated by reference and were set forth in its entirety herein.

1. A CD83 binding protein comprising the amino acid sequences of SEQ ID NO: 1, SEQ ID NO: 23, and SEQ ID NO: 24.

2. The CD83 binding protein of claim 1, wherein the binding protein comprises the amino acid sequences of SEQ ID NOs: 1-3 or SEQ ID NOs: 11-13.

3. (canceled)

4. The CD83 binding protein of claim 1, wherein the binding protein comprises the amino acid sequence of SEQ ID NO: 25.

5. The CD83 binding protein of claim 4, wherein the binding protein comprises the amino acid sequence of SEQ ID NO: 4 or SEQ ID NO: 14.

6. (canceled)

7. The CD83 binding protein of claim 1, wherein the binding protein comprises the amino acid sequence of SEQ ID NO: 5.

8. The CD83 binding protein of claim 1, wherein the binding protein comprises the amino acid sequence of SEQ ID NO: 26.

9. The CD83 binding protein of claim 8, wherein the binding protein comprises the amino acid sequence of SEQ ID NO: 6 or SEQ ID NO: 16.

10. (canceled)

11. The CD83 binding protein of claim 1, wherein the binding protein comprises the amino acid sequence of SEQ ID NO: 7.

12. The CD83 binding protein of claim 1, wherein the binding protein comprises the amino acid sequence of SEQ ID NO: 8.

13. The CD83 binding protein of claim 12, wherein the binding protein comprises the amino acid sequence of SEQ ID NO: 9.

14. The CD83 binding protein of claim 1, wherein the binding protein comprises the amino acid sequence of SEQ ID NO: 18.

15. The CD83 binding protein of claim 14, wherein the binding protein comprises the amino acid sequence of SEQ ID NO: 19.

16. A nucleic acid molecule comprising a nucleotide sequence encoding the CD83 binding protein of claim 1.

17. (canceled)

18. A method of making a CD83 binding protein, the method comprising culturing a host cell comprising the nucleic acid molecule of claim 16 under conditions which allow expression, and recovering the CD83 binding protein.

19. (canceled)

20. A multispecific construct comprising an NK cell engaging domain, an NK activating domain operably linked to the NK engaging domain, and a targeting domain comprising the CD83 binding protein of claim 1 operably linked to the NK activating domain and the NK engaging domain.

21.-38. (canceled)

39. A chimeric antigen receptor (CAR) comprising an extracellular domain comprising the CD83 binding protein of claim 1, a transmembrane domain, and an intracellular signaling domain.

40.-49. (canceled)

50. A method of suppressing alloreactive donor cells in a subject receiving transplant donor cells, the method comprising administering to the subject an effective amount of the immune effector cell of claim 46.

51.-52. (canceled)

53. A method of treating cancer in a subject, the method comprising administering to the subject an effective amount of the immune effector cell of claim 46.

54.-57. (canceled)

58. A method of treating an alloimmune condition or an autoimmune condition in a subject in need thereof, the method comprising administering to the subject an effective amount of the immune effector cell of claim 46.

59.-60. (canceled)
